(19) United States
(12) Patent Application Publication
(10) Pub. No.: US 2006/0099619 A1
(43) Pub. Date: May 11, 2006

(54) Detection and quantification of miRNA on microarrays

(75) Inventors: Jose Remacle, Malonne (BE); Sandrine Hamels, (US); Francoise du Longueville, Natoye (BE)

Correspondence Address: FOLEY AND LARDNER LLP;SUITE 500, 3000 K STREET NW, WASHINGTON, DC, 20007, US

(73) Assignee: Eppenddorf Array Technologies, S.A.

(21) Appl. No.: 11/242,139

(22) Filed: Oct. 4, 2005

Related U.S. Application Data
(63) Continuation-in-part of application No. 10/637,656, filed on Aug. 11, 2003.

(51) Int. Cl. C12Q 1/68; C12P 19/34

(52) U.S. Cl. 435/6; 435/91.2

(57) ABSTRACT

The present invention relates to a new method for the detection, identification and/or quantification of multiple gene-specific mRNA or stRNA, respectively, the inducers of RNAi. In particular the present invention relates to a method for detecting the presence or change in concentration of mRNA in a cell, which change may be induced by environmental conditions.

### FIELD OF THE INVENTION

The present invention relates to a method for the determination of the cellular transcriptional regulation based on a simultaneous detection and quantification of a pattern of mRNAs, being part of the RNAi, in a cell.

### DESCRIPTION OF THE RELATED ART

In experiments, during which dsRNA was injected into the nematode Caenorhabditis elegans it was found that a silencing of genes highly homologous in sequence to the delivered dsRNA occurred (Fire et al., Nature 391 (1998), 806-811). Based on this finding the term “RNA interference” (RNAi) was created nominating the capability of such dsRNA-molecules to affect the translation of transcripts.

During ensuing research in this area it has been shown that dsRNA triggers degradation of homologous RNAs within the region of identity with the dsRNA (Zamore et al., Cell 101 (2000), 25-33). Apparently, the dsRNA is processed to RNA fragments exhibiting a length of about 21-23-ribonucleotides (Zamore et al., supra). These short fragments were also detected in extracts prepared from Drosophila melanogaster Schneider 2 cells that were transfected with dsRNA before cell lysis (Hammond et al., Nature 404 (2000), 293-296) or after injection of radiolabelled dsRNA into D. melanogaster embryos (Yang et al., Curr. Biol. 10 (2000), 1191-1200) or C. elegans adults (Parrish et al., Mol. Cell 6 (2000), 1077-1087).

RNAi was observed to also be naturally present in a wide range of living cells. For example, these kind of molecules have been found to exist in insects (Kennerdell and Carthew, Cell 95 (1998), 1017-1026), frog (Oelgeschlager et al., Nature 405 (2000), 757-763), and other animals including mice (Svoboda et al., Development 127 (2000), 4147-4156; Wianny and Zemicka-Goetz, Nat. Cell Biol. 2 (2000), 70-75) and also in humans. RNA molecules of similar size have also been found to accumulate in plant tissue that exhibits post-transcriptional gene-silencing (PTGS) (Hamilton and Baulcombe, Sciences 286 (1999), 950-952).

RNAi is closely linked to the post-transcriptional gene-silencing (PTGS) mechanism of co-suppression in plants and quelling in fungi (Cogoni and Macino, Curr. Opin. Microbiol. 2 (1999), 657-662; Catalanotto et al., Nature 404 (2000), 245; Dalmay et al., Cell 101 (2000), 543-553; Ketting and Plasterk, Nature 404 (2000), 296-298; Mourrain et al., Cell 101 (2000), 533-542; Smardon et al., Curr. Biol. 10 (2000), 169-178), and some components of the RNAi machinery are also necessary for post-transcriptional silencing by co-suppression (Catalanotto et al., Nature 404 (2000), 245; Dernburg et al., Genes & Dev. 14 (2000), 1578-1583; Ketting and Plasterk, Nature 404 (2000), 296-298).

The natural function of RNAi and co-suppression appears to be protection of the genome against invasion by mobile genetic elements, such as transposons and viruses, which produce aberrant RNA or dsRNA in the host cell when they become active (Jensen et al., Nat. Genet. 21 (1999), 209-212; Ketting et al., Cell 99 (1999), 133-141; Ratcliff et al., Plant Cell 11 (1999) 1207-1216; Tabara et al., Cell 99 (1999), 123-132; Malinsky et al., Genetics 156 (2000), 1147-1155). Specific mRNA degradation prevents transposon and virus replication, although some viruses seem to be able to overcome or prevent this process by expressing proteins that suppress PTGS (Anandalakshmi et al., Science 290 (2000), 142-144; Lucy et al., EMBO J. 19 (2000), 1672-1680; Voinnet et al., Cell 103 (2000), 153-167).

The currently existing model for the mechanism of RNAi is based on the observation that the introduced dsRNA is bound and cleaved by RNase III-like enzyme Dicer to generate products having the length indicated above. These molecules, termed small interfering RNAs (siRNAs) trigger the formation of RNA-induced silencing complex (RISC). The resulting dsRNA-protein complexes appear to represent the active effectors of selective degradation of homologous mRNA (Hamilton and Baulcombe, Sciences 286 (1999), 950-952, Zamore et al., Cell 101 (2000), 25-33; Elbashir et al., Genes & Dev. 15 (2001), 188-200.) Elbashir et al. provide evidence that the direction of dsRNA processing determines whether sense or antisense target RNA can be cleaved by the siRNA-protein complex. Helicases in the complex unwind the dsRNA, and the resulting single-stranded RNA (ssRNA) seems to be used as a guide for substrate selection. Once the ssRNA is base-paired with the target mRNA, a nuclease activity, presumably within the complex, degrades the mRNA.

The DICER enzyme which produces the siRNA also produces other types of small RNA molecules termed microRNA (mRNA). These miRNA are processed from endogenous transcripts that form hairpin structures. The miRNA formed are involved in the control of other genes by binding to the 3′ end of their messenger RNA in animals (Chi et al, Proc. Natl. Acad. Sci. 100 (2003), 6343-6346).

Both miRNA and siRNA are part of the RNAi and they are processed by the DICER enzyme complex in order to produce small double stranded RNA with non frank end and a phospate at the 5′end of each strand. The mode of action of the RNAi in the RISC complex (RNA-Induced Silencing Complexes) is the same for both RNAi and depends on the fact that there is or not a perfect match between the siRNA or the miRNA and the mRNA on which they hybridized. If the match is perfect, the RISC-RNA complex degrades the targeted mRNA with a concurrent cleavage and degradation of the mRNA. If there is mismatch, the translation of the target mRNA reading in the ribosome is repressed and the protein is not synthetized. So both molecules are the actors of the RNAi process with similar mode of action even if they differ in their biological role. Recently a distinction has been made between siRNA and miRNA, both of which molecules have the same structure and may act in the same way.

Thus, RNAi seems to be an evolutionary conserved mechanism in both plant and animal cells that directs the degradation of mRNA homologous by miRNA. The ability of mi RNA to direct gene silencing in mammalian cells contitute a new level of regulation of the transcription and is thus essential to understand the role of this new level of regulation on the cell response to external or internal stimuli. Also the understandinng of the role of specific miRNA on gene silencing in mammalian cells has raised the possibility that miRNA might be used as siRNA to investigate gene function in a high throughput fashion or to specifically modulate gene expression in human diseases

In human, there are between 200 and 300 miRNA genes and about 200 have been identified at the moment. In heart, liver or brain, it is found that a single, tissue-specifically expressed miRNA dominates the population of expressed miRNAs and suggests a role for these miRNAs in tissue specification or cell lineage decisions (Lagos-Quintana et al. Current Biology 12 (2002), 735-739).

Characterization of a number of miRNAs indicates that they influence a variety of processes, including early development (Reinhart et al. Nature 403 (2000), 901-906), cell proliferation and cell death (Brennecke et al. Cell 113 (2003), 25-36), and apoptosis and fat metabolism (Xu et al. Curr. Biol. 13 (2003), 790-795). In addition, one study shows a strong correlation between reduced expression of two miRNAs and chronic lymphocytic leukemia, providing a possible link between miRNAs and cancer (Calin et al., Proc Natl Acad Sci USA 99 (2002), 15524-15529). Although the field is still young, there is speculation that miRNAs could be as important as transcription factors in regulating gene expression in higher eukaryotes.

miRNAs affects the expression of target genes by one of at least two mechanisms. Some bind to the 3′UTR of target mRNAs and suppress translation (Chi et al., Proc Natl Acad Sci USA. 100 (2003), 6343-6346). Others act as siRNAs, binding to and destroying target transcripts. miRNAs interfere with expression of messenger RNAs encoding factors that control developmental timing, stem cell maintenance, and other developmental and physiological processes in plants and animals. miRNAs are negative regulators that function as specific determinants, or guides, within complexes that inhibit protein synthesis (animals) or promote degradation (plants) of mRNA targets (Carrington and Ambros, Science. 301 (2003), 336-338). Plants with altered miRNA metabolism have pleiotropic developmental defects. In Arabidopsis, a miRNA has been identified “JAW” that can guide messenger RNA cleavage of several TCP genes controlling leaf development (Palatnik et al., Nature 425 (2003), 257-263).

Recently, miRNAs have been identified in undifferentiated and differentiated mouse embryonic stem (ES) cells (Houbaviy et al. Dev Cell 5 (2003), 351-358). Their expression is repressed as ES cells differentiate into embryoid bodies and is undetectable in adult mouse organs. In contrast, the levels of many previously described miRNAs remain constant or increase upon differentiation. These results suggest that miRNAs may have a role in the maintenance of a pluripotent cell state and in the regulation of early mammalian development.

Finally, miRNA mechanism of action is diverse and does not only target RNA transcript. miRNA's may also regulate gene expression by causing chromatin condensation. Several groups have shown that binding of dsRNAs to plant-promoter regions can cause gene silencing—an effect that is mediated via DNA methylation.

The detection of naturally occurring miRNA is difficult to perform given the large number of molecules, their small size and their low number in the cells. Also the method has o provide quantitative assay of the different miRNA. None of the previously cited documents provide an easy method for detecting and analyzing naturally occurring miRNA, the inducer of RNAi. One method which has been proposed is based on the cloning of miRNAs after addition of linker segments to their 5′- and 3′-termini using T4 ligase and amplification of the elongated RNA (Elbashir et al., Gene & Dev. 15 (2001), 188-200). The analysis of the cloned fragments was performed by sequencing. As only one miRNA can be evaluated at a time, this method is very time consuming and expensive.

Trancriptional regulation of multiple gene expression is a complex and subtle process. In order to investigate the effect of the miRNA on their transcribed genes, the assay has to be quantitative and multiple since small variation in their amount affects the gene expression in a significant way and modifies the cell composition.

Thus, there is a need in the art for a sensitive method to determine, whether a cell is subject to a RNAi mediated transcriptional regulation provided by the miRNA.

### SUMMARY OF THE INVENTION

An object of the present invention is to provide a method and tools for rapidly and reliably detecting and quantifying the cellular transcriptional regulation mediated by RNAi due to the presence of naturally occuring miRNAs.

In accomplishing these and other objects of the invention, there is provided, in accordance with one aspect of the invention, a method for detecting a miRNA directed against at least one specific gene present in a sample comprising the steps of: (i) isolating miRNA from a target cell; (ii) contacting the miRNA with an array of capture probes under hybridization conditions; and (iii) detecting a signal or a change in a signal on the array.

There is also provided, in accordance with another aspect of the invention, a method for detecting multiple miRNA directed against specific gene present in a sample comprising the steps of: (i) isolating miRNA from a target cell; (ii) contacting the miRNA with an array of at least 3 capture probes arranged in specific locations under hybridization conditions; and (iii) detecting and quantifying a signal or a change in a signal in the specific locations of the array. The inventive method further comprises the step of elongating or ligating said miRNAs into target labeled polynucleotides. The method also comprises possible labelling and/or enzymatically copying the miRNA prior to contact with the array.

The RNAi mediated cellular transcriptional regulation is provided by the detection and quantification of a pattern of miRNA.

In one embodiment, the cell transcriptional regulation provided by the detection and quantification of a pattern of miRNAs is correlated with the pattern of expression of the genes having mRNA sequences complementary to the corresponding miRNA sequences detected in the same sample. In another embodiment, the RNAi mediated cellular transcriptional regulation is provided by the detection and quantification of a pattern of miRNAs is correlated with the pattern of expression of the genes having mRNA sequences having more than 90% homology to the corresponding miRNA sequences in the same sample.

In another embodiment, the detected miRNAs are mature miRNAs. In another embodiment, the invention provides a method, wherein the cellular transcriptional regulation is related to one of the following fields: development, cell differentiation or stem cell maintenance, cell proliferation, cell death, chromatin condensation or cell transformation.

In one embodiment, the detection of the miRNA is performed after elongation of the miRNA on one of its complementary sequences. In another, each capture probe contains at least one label. In this embodiment, RNase H can be used to release the label from the capture probe after the capture probe binds the miRNA.

In an alternative embodiment, the DNA/DNA-RNA hybrid complex obtained by elongation is then amplified by any linear amplification methods such as in vitro RNA transcription, asymetric or linear PCR. In a preferred embodiment, one primer is provided for linear amplification of the elongated sequences. Quantification of the multiple miRNA present in a sample is provided by one simple treatment of all the miRNA and direct hybridization on their corresponding capture

In another embodiment, the detection of the miRNA is performed after ligation of the miRNA hybridized on its complementary bait sequence with an adjacent probe. In another embodiment, the adjacent probe is pre-hybridized with its complementary bait sequence before ligation with the miRNA. In still another embodiment, the T4 RNA ligase may be used for carrying out the ligation reaction. In a preferred embodiment, the adjacent probe is labeled.

In another embodiment, the detection of the miRNA is performed after elongation of the miRNA by tailing and labelling with a mixture of labeled ATP and unlabeled ATP using poly(A) polymerase. If biotine is used as label, then the tailing and labelling, the 3′ extremity of the miRNA is biotinylated labelled. The labelled miRNA are then hybridized to their complementary probes. The detection of hybrid is performed by an incubation of anti-biotin antibody coupled with fluorochrome Cy3 or using the Silverquant detection method (Eppendorf, Hamburg, Germany).

The invention further provides kits for the determination of cellular transcriptional regulation in a sample comprising an array comprising capture probes being arranged in specific locations and having sequences identical or complementary to miRNAs of interest or parts thereof and optionally, buffers and labels. In another embodiment, the kit may also comprise a second array for the detection and quantification of the expression of the regulated genes in the same sample.

Other objects, features and advantages of the present invention will become apparent from the following detailed description. The detailed description and specific example, while indicating preferred embodiments, are given for illustrative purposes only, since various changes and modifications within the spirit and scope of the invention will become apparent to those skilled in the art from this detailed description. Further, the example demonstrates the principle of the invention and cannot be expected to specifically illustrate the application of this invention to all the examples where it will be obviously useful to those skilled in the prior art.

In still another embodiment, the inventive methods can be used to identify compounds useful in regulating gene transcription.

The invention further provides kits for detecting miRNA directed against at least one gene present in a sample comprising an array comprising capture probes positioned at specific locations and having sequences at least 90% homologous to mRNAs of interest or parts thereof and optionally, buffers and labels.

Also provided is a screening device for testing the effect of compounds on the presence of miRNA directed against at least one gene, said screening device comprising an array comprising capture probes positioned at specific locations and having sequences at least 90% homologous to mRNAs of interest or parts thereof and optionally, buffers and labels.

In another embodiment, the present invention provides a method based on the use of micro-arrays for the specific detection of the miRNA molecules directed to specific genes or family of genes and being present in cells or cell extracts.

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### DESCRIPTION OF THE DRAWINGS

FIG. 1 shows embodiment for the detection of miRNAs in which they are first incubated in solution with their complementary DNA strands and after elongation and labeling, are detected by hybridization on array bearing sequences complementary to the miRNA and/or of its elongated target labeled polynucleotide.

FIG. 2 shows embodiment where the miRNA is elongated to form a priming sequence that is used with a complementary primer for linear amplification using polymerases. The amplified labeled amplicons are then detected on a micro-array bearing sequences complementary to the miRNA and/or of its elongated .target labeled polynucleotide.

FIG. 3 presents the labelling of the miRNA obtained by ligation with an adjacent labelled probe. After denaturation, labelled strands are used for incubation with capture probes present on the array.

FIG. 4 shows an embodiment for detection of miRNA by linear amplification of baits using rolling circle amplification. A pool of single stranded circular baits targeting one or more miRNA are hybridized in solution to the miRNA sample preparation. The annealed miRNAs then act as RNA primers for selected DNA-dependent DNA polymerases to initiate DNA synthesis on the miRNA-primed bait template molecule. The polymerase elongation is performend in the presence of labelled nucleotides. The RNA-primed bait-DNA polymerase reaction is further subjected to a second DNA polymerase with strong strand displacement activity to transform the initial primer extension reaction into a rolling circle amplification synthesis. The long single-stranded DNA molecules comprising DNA concatemers of miRNA sequences is fragmented to miDNA monomers to facilitate hybridisation with capture probes in the array. The fragmentation is achieved in a sequence-specific manner by hybridization to DNA-oligonucleotides having a length between 6 and 15 and preferably between 9 and 12, which are complementary to a unique restriction endonuclease site placed downstream of the miRNA sequence on the bait DNA, followed by incubation with the corresponding restriction endonuclease. The miRNA specific polynucleotides are detected on a microarray presenting capture probes complementary to the amplified product.

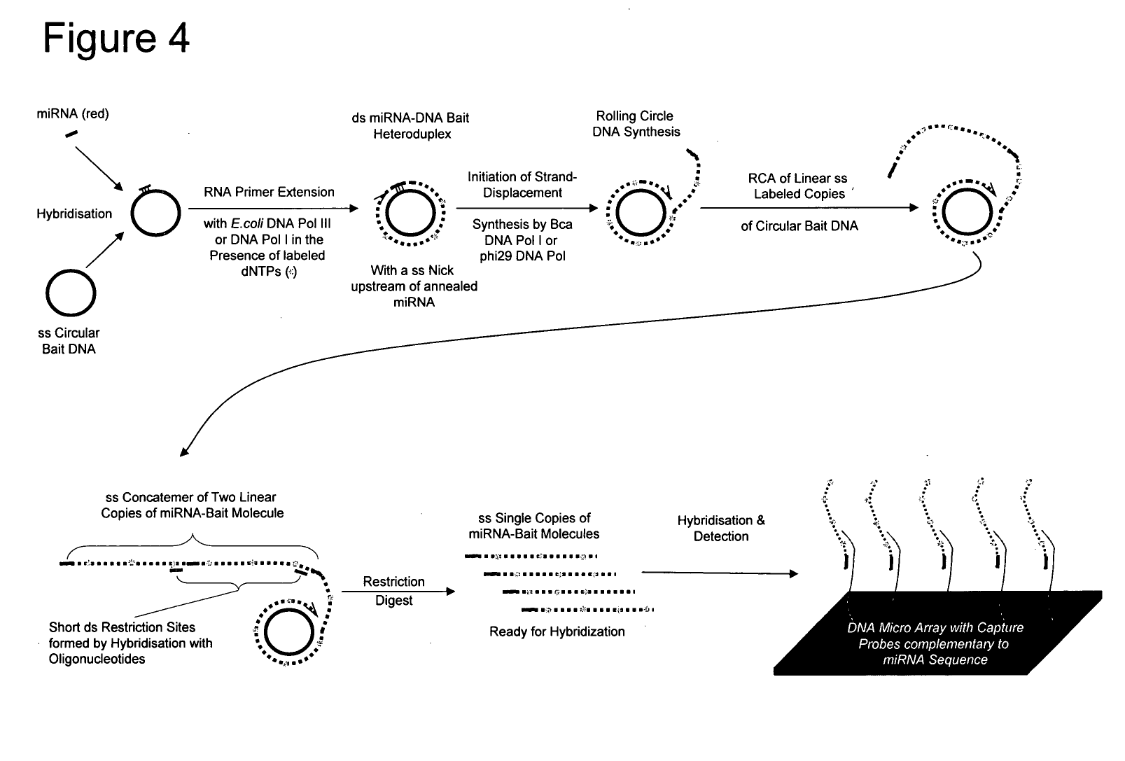

FIG. 5 shows a preferred embodiment where the miRNA is tailed and labelled with a mixture of biotinylated ribonucleotides and unlabelled ribonucleotides using poly A polymerase. Labelled miRNAs are then detected by hybridization on array bearing sequences complementary to at least part of the sequence of the miRNA. The biotinylated hybrids are detected after reaction with Cy3 labeled anti-biotin antibodies.

FIG. 6 presents the detection and quantification of miRNA on array in brain tissue according to the method described in FIG. 5 and the miRNA sequences are presented in Table 1.

### DETAILED DESCRIPTION OF THE INVENTION

Definitions

The term “genes” shall designate the genomic DNA which is transcribed into mRNA and then translated into a peptides or proteins. The measurement of the expressed genes is performed on either molecules within this process most currently the detection of the mRNA or of the peptide or protein. The detection can also be based on specific property of the protein being for example its enzymatic activity.

The terms “nucleic acid, array, probe, target nucleic acid, bind substantially, hybridizing specifically to, background, quantifying” are as described in the international patent application W097/27317, which is incorporated herein by reference.

The term “nucleotide triphosphate” refers to nucleotides present in either as DNA or RNA and thus includes nucleotides which incorporate adenine, cytosine, guanine, thymine and uracil as bases, the sugar moieties being deoxyribose or ribose. Other modified bases capable of base pairing with one of the conventional bases adenine, cytosine, guanine, thymine and uracil may be employed. Such modified bases include for example 8-azaguanine and hypoxanthine.

The term “nucleotide” as used herein refers to nucleotides present in nucleic acids (either DNA or RNA) compared with the bases of said nucleic acid, and includes nucleotides comprising usual or modified bases as above described.

References to nucleotide(s), oligonucleotide(s), polynucleotide(s) and the like include analogous species wherein the sugar-phosphate backbone is modified and/or replaced, provided that its hybridization properties are not destroyed. By way of example, the backbone may be replaced by an equivalent synthetic peptide, called Peptide Nucleic Acid (PNA).

The terms “nucleotide species” is a composition of related nucleotides for the detection of a given sequence by base pairing hybridization; nucleotides are synthesized either chemically or enzymatically but the synthesis is not always perfect and the main sequence is contaminated by other related sequences like shorter one or sequences differing by a one or a few nucleotides. The essential characteristic of one nucleotides species for the invention being that the overall species can be used for capture of a given sequence.

“Polynucleotide” sequences that are complementary to one or more of the miRNA described herein, refers to polynucleotides that are capable of hybridizing under stringent conditions to at least part of the nucleotide sequence of said RNA or RNA copies. Given the small size of the miRNA, the capture molecules have to be identical or at least have more than 90% identical sequence in order to specifically detect the miRNA beside other possible flanking regions.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The term “capture probe” refers to a polynucleotide which specifically binds to another polynucleotide corresponding to a gene and/or transcript of a cell of interest. Polynucleotide binding is obtained through base pairing between the two polynucleotides, one being the immobilized capture probe and the other one the target to be detected.

The term miRNA is a non coding small RNA produced by a DICR enzyme from a double stranded RNA Precursor. The precursor has a stem loop or hair-pin structure. miRNA are present in animals or plants. They can bind to a protein complex termed miRISCs. They represent one of the components of the RNAi beside other ones like the siRNA.

The present invention is based on the use of arrays having multiple single nucleotide sequences arranged in specific, locations thereon and being identical or complementary to miRNA present in the cells for which the miRNA are to be determined.

In one preferred embodiment the present invention provides an arrays having multiple single nucleotide sequences arranged in specific, locations thereon and being identical or complementary to miRNA, present in the cells for which the pattern of transcriptional regulation is to be determined.

In one particular embodiment, the array comprises 5-500 and preferably 20-5000 capture probes.

One preferred embodiment of the invention is to obtain a pattern of transcriptional regulation based on the simultaneous detection and quantification of multiple miRNAs present in a cell. The signals of the different spots related to each gene being a direct measurement of the diversity and the concentration of the miRNA in the analysed cells or tissues. Also, the invention is not limited by the number of miRNA to be screened. The array allows to analyse either from 5 to 500 and more preferably until 5000 miRNAs in a cell. This number depends on the species and the number of expressed miRNA genes in the analysed cells.

The present invention provides a method for the determination of cellular transcriptional regulation by the simultaneous detection and quantification of multiple miRNAs present in a cell on an array and by detecting a signal present on a specific location on the array, said signal at such location being related to the presence of one miRNA with the detection of at least 3, preferably at least 5, more preferably at least 10 and even more preferably at least 20 miRNAs on the array being indicative of a given miRNA or RNAi mediated cellular transcriptional regulation.

In general, in a cell, there are typically about 20 miRNA genes expressed. In human there are about 200 to 300 miRNA genes. The identification of a pattern of expressed miRNAs in a given cell brings an answer to the question, whether a cell is subject to RNAi mediated transcriptional regulation (e.g. the genes regulated by these miRNA and their target genes). In a preferred embodiment, to unravel the cellular transcriptional regulation, the pattern of at least 3 miRNAs obtained by the method of the invention is correlated with the pattern of expression of the regulated genes in the same sample (e.g provided by a second array). In another embodiment, the pattern of at least 3 miRNA is correlated with the pattern of expression of the miRNA target genes in the same sample (e.g provided by a second array). In an alternative embodiment, the pattern of at least 3 miRNA is correlated with the pattern of expression of genes having mRNA sequences at least 90% homologous to the corresponding miRNA sequence in the same sample (e.g. provided by a second array). In another embodiment, the pattern of at least 3 miRNAs obtained by the method of the invention is correlated with activated transcriptional factors in the same sample.

In a preferred embodiment, the invention provides a method for the simultaneous detection of at least 3 and preferably 20 and even preferably 50 of the miRNA presented in Table 1 for human cells and at least 3 miRNA and preferably 20 and even preferably 50 presented in Table 2 for mouse cells.

In a preferred embodiment, the invention provides a method for the simultaneous detection of at least 3 and preferably 5 and even preferably 10 of the miRNA presented in Table 3 for human cells. Each individually detected miRNA from Table 3 regulates one or several targeted genes. A list of miRNA sequences and their targeted genes are available www.microrna.org. The present invention covers the detection of part or all of the miRNA presented in this publication and data bank.

In a preferred embodiment, the invention provides a method wherein the cellular transcriptional regulation is related to one the following fields: development, cell differentiation or stem cell maintenance.

Preferably the capture probes contain at least part of their sequence being complementary of the miRNA and having between 15 and 25 bases and even preferably between 19 and 23 bases. Preferably the specific part of the capture probe sequence have Tm comprised between 54 and 72° C. and preferably between 62 and 66° C.

Preferably the specific sequence is provided at the end of a spacer being preferably located at a distance of 6.8 nm from the support and even preferably being a sequence of nucleotides being at least 20 bases and preferably more than 90 bases.

The support is generically composed of a solid surface which may be selected from the group consisting of glasses, electronic devices, silicon supports, silica, metal or mixtures thereof prepared in format selected from the group of slides, discs, gel layers and/or beads. Beads are considered as arrays in the context of the present invention, as long as they have characteristics which allow a differentiation from each other, so that identification of the beads is correlated with the identification of a given capture probe and so of the target sequence.

On the support, a number of capture molecules are fixed by covalent binding, each capture molecule being located at a specific location and having at least in part a sequence in a single strand form complementary to the miRNA to be screened. Preferably the array comprises capture probes ranging from 10 to 1000 nucleotides, preferably from about 15 to 200, or 15 to 100 nucleotides. The array preferably comprises between 5-1000 and still preferably 50-300 different capture probes.

Generally, the capture probes may be synthesized by a variety of different techniques, but are preferably by chemical synthesis or by PCR amplification from cloned genes using an aminated primer.

The amino group of the amplicon is then reacted with a functionalized surface bearing reactive groups, such as, but not limited, to aldehyde, epoxide, acrylate, thiocyanate, N-hydroxysuccinimide. After having formed a covalent linkage, the second strand of the amplicon is then removed by heating or by alkaline treatment so that single strand DNA or RNA is present on the surface and ready to bind to the complementary siRNA or siRNA copies.

Given the progress of chemical synthesis of the nucleotides, the use of chemically synthesised polynucleotides is also envisaged in the invention. The synthesised nucleotides are also preferably aminated or thiolated and deposited on the functionalized surface. Advantage of the chemically synthesised nucleotides is their ease of production.

Methods of arranging nucleotides and polynucleotides are well known in the art and may be found in Bowtell, D. and Sambrook (DNA Microarrays, J. Cold Spring Harbor Laboratory Press, 2003 Cold Spring Harbor, N.Y., pg 1-712) which is incorporated herein by reference. In a preferred embodiment the nucleotide sequence is attached to the support via a linker, which may be a polynucleotide exhibiting a length of between about 20 to 200 nucleotides (EP 1 266 034). In principle, the capture probes may be DNA, PNA or RNA.

In one step of the method (step (ii)), miRNA from a cell of interest is isolated. An exemplary process for the isolation of small interference RNA (siRNA) is described e.g. in Tuschl et al. (Genes & Dev. 13 (1999), 3191-3197), which document is incorporated herein by way of reference.

The labelling is preferably performed by attaching a specific molecule to the miRNA or one of its copy of a derivative thereof, that is detected, e.g. via fluorescence, colorimetry, chemo- or bioluminescence, electric, magnetic or particularly biotin. Indirect labelling is also of used when amplification of the signal is required. Biotin-labelled nucleotides is one of the preferred molecule attached/incorporated, which is then recognized by binding proteins being either antibodies or streptavidin or related binding molecules. The binding proteins are labelled by any chemical or physical means and detected and quantified.

In an alternative embodiment, the capture probes present on the array may contain a label at their 3′-end. After binding of the miRNA, the RNA/DNA hybrids are then cleaved with RNase H thus releasing the label from those capture probes, where the miRNA had bound. Therefore, in this embodiment, the decrease in signal is representative of the presence of a miRNA present in the sample.

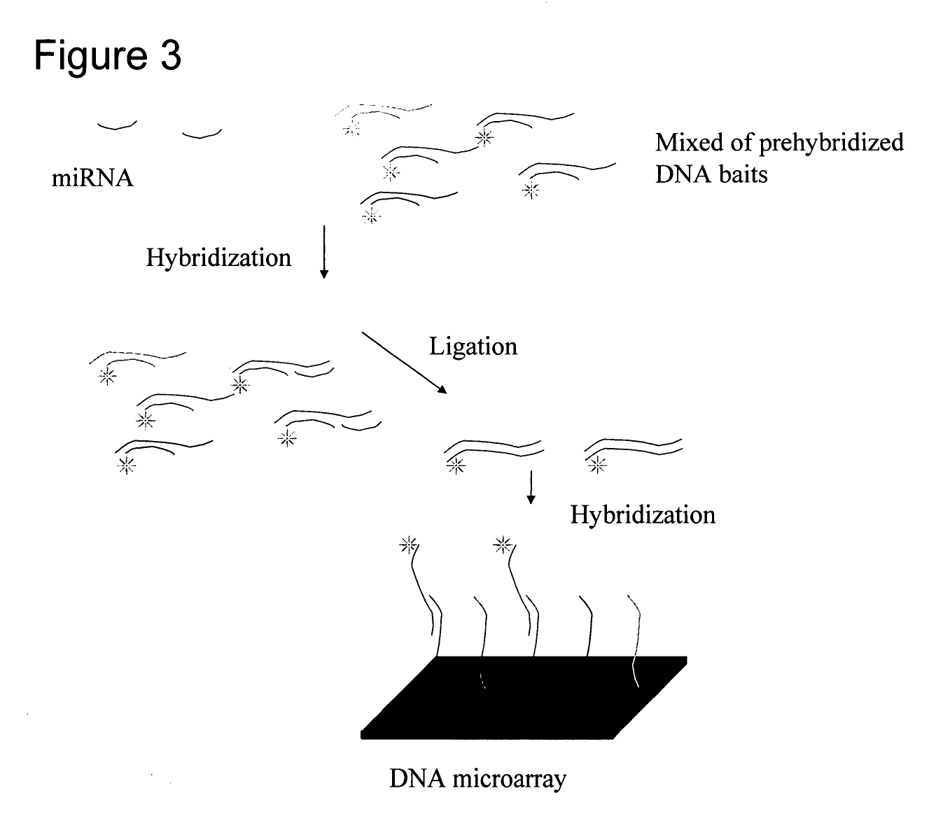

In an first embodiment, the released fragments is preferably detected and/or quantified and/or identified by their hybridization on specific capture probes present on a second DNA microarray (cf. FIG. 3). In a second embodiment, the released fragments are separated, identified and/or quantified after electrophoresis. The size of the released fragments indicates the location of binding of the miRNA and allow their identification. Also a sequence analysis of released sequence will lead to the same identification (FIG. 3).

The miRNA is also preferably transcribed to their corresponding DNA-copies or amplified by means of PCR. Accordingly, the copying is performed using a retro-transcriptase allowing for the incorporation of labelled nucleotides in the forming strand. Also, the miRNA is subjected to a PCR-reaction, which in principle involves the use of 3′- and 5′-adapter oligonucleotides in order to perform a blunt end ligation with the multiple extracted miRNAs in solution. The product thus obtained is then reverse transcribed with a 3′-RT primer complementary to the 3′-adapter. Subsequently, a PCR amplification cycle is then perform with a 5′-primer complementary of the cDNA and in the presence of the 3′-RT primer. Labeled nucleotides are incorporated into the amplicons during the PCR-reaction.

In a preferred embodiment, the labeling is performed by incubating the miRNA with a mixture of ssDNA under conditions as to obtain formation of a RNA-DNA hybrid complex, whereupon an elongation and concurrent labeling of the small miRNA is achieved. Here, the ssDNA bait is used as a matrix and labeled ribonucleotides/deoxynucleotides are utilized for the elongation of miRNA.

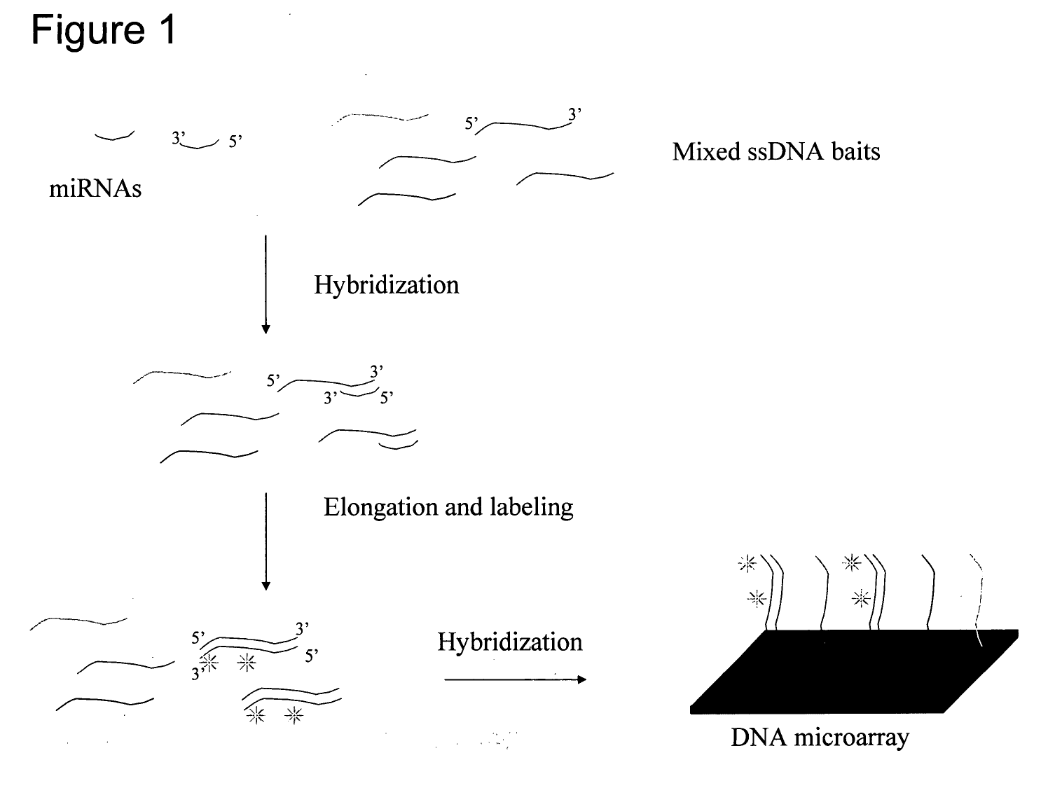

The ssDNA bait used for the formation of the hybrid complex is replaced by any nucleotide or nucleotide-like molecules as long as the elongation of the bound miRNA is possible. After denaturation, the labeled strand will be used for incubation with the capture probes present on the array for detection and quantification of the miRNA (FIG. 1).

According to a preferred embodiment, the elongation is performed with the Tth DNA polymerase 3 which accept as primer RNA sequences such as miRNAs. The elongated and labeled polynucleotide is DNA. In a particular embodiment the elongation of the miRNA is performed by tailing the miRNA using the PolyA polymerase.

In a preferred embodiment, the ssDNA bait is a sequence complementary to the corresponding miRNA (−) for which the analysis is required. After elongation and labeling, the elongated strand (−) is hybridized on a capture probe (+) identical to the mRNA strand or part of it. The same capture probe may be used for parallel detection and quantification of the mRNA present in the same sample. After retro-transcription of the mRNA (+), the labeled cDNA (−) is hybridized on the same capture probe. This method greatly simplifies the production of the capture probes which are equivalent for both applications.

In another embodiment, detection of the miRNA and their precursor is accomplished by providing a specific synthetic bait DNA polynucleotide during a labelling reaction using the complete DNA polymerase I, E. coli DNA polymerase III or Tth DNA polymerase III holoenzyme. The RNA nucleotides complementary to the DNA baits serve as primers for the DNA polymerase extension. The bait is designed to bind either the one of the miRNA strands or to a nucleotide sequence exclusively present in the precursor forms of miRNA.

This bait contains further a nucleotide extension allowing for incorporation of multiple labelled nucleotides and contain in its 3′ end a series of nucleotides that serve as complements to the microarray capture probe. The labelled nucleotide incorporation is maximised by using an optimized sequence composition allowing for multiple labelled nucleotides to be incorporated with high efficiency. The 3′ end of the bait is designed with a sequence tag that is unique for each RNA molecule and hybridize to a complementary capture probe of the microarray. In this case, the array is a standard array of barcode tagged capture probes, and the specificity is provided by the bait in the labelling step. Baits are designed with a nucleotide sequence specific for each detection application. The same enhancement strategy using LNA can be used.

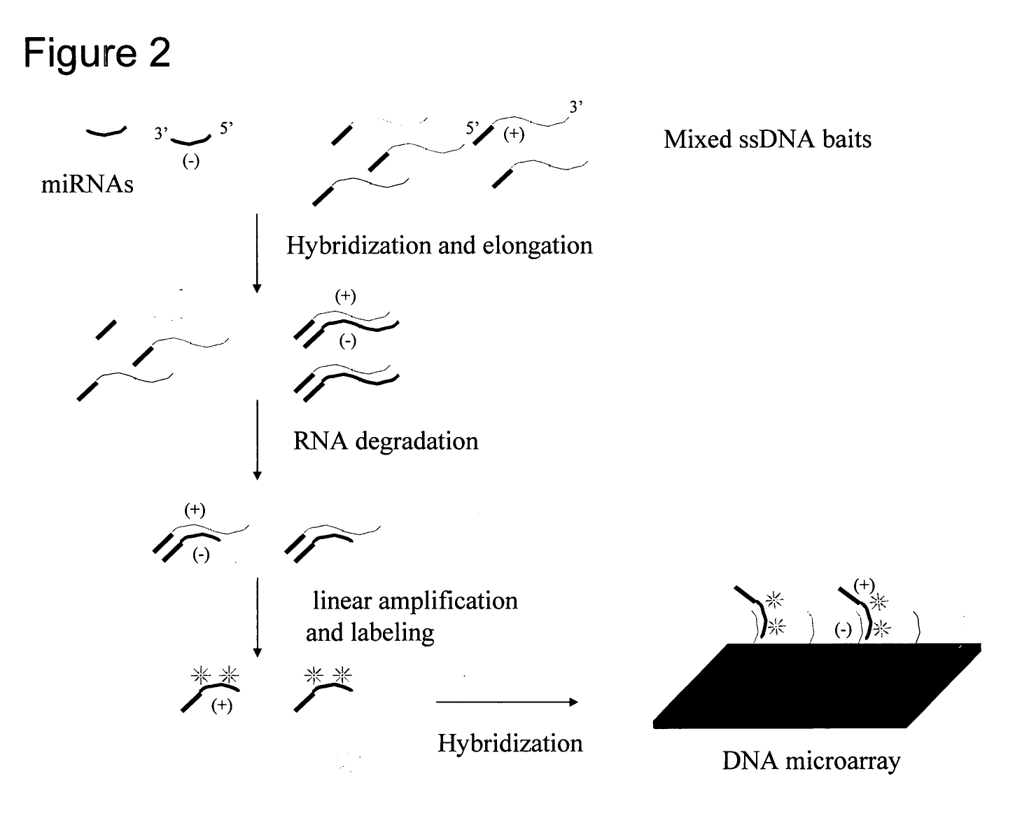

In a preferred embodiment, the mixture of ssDNA baits is composed of three parts: the 3′ end is complementary of the miRNA, the middle part is specific of each bait and the 5′ end sequence is common to all baits. After hybridization of the miRNA and their elongation, the product is amplified. After degradation of the miRNA, the matrix for the amplification is a DNA/DNA hybrid complex. A primer complementary of the common sequence of the elongated DNA is provided for linear amplification with a DNA polymerase. Advantageously, only one primer is required for the amplification of all elongated miRNAs. Altered cycles of denaturation, primer annealing and polymerisation are performed like in a normal PCR except that only one primer is used which results in a linear amplification. The advantage of such amplification is that quantification of the initial amount of miRNA remains possible due to the linearity of the amplification. After linear amplification, the products are detected on an array bearing sequences complementary at least partly to the amplified product (FIG. 2). The target labeled nucleotides which are hybridized on the array are preferably labeled during the amplification. Preferably, the capture probes of the array do not comprise the primer sequence used for the amplification nor the miRNA sequences or their complement. In order to avoid interference between the ssDNA baits (+) introduced at the beginning of the assay with the amplified labeled products (+) for the hybridization on the capture probes (−) of the array, the mixture of ss DNA baits may be specifically degraded before the amplification (e.g. by S1 nuclease).

Alternatively, the primer complementary of the common sequence of the elongated DNA comprises a T7 promoter sequence for an RNA polymerase that might be used for in vitro transcription. The primer may also comprise a Tag sequence which is used for further amplification (e.g. the tag may be a sequence rich in cytosine if the amplification is performed with labeled CTP, thus increasing the number of incorporated label during the amplification).

The labeling is preferably obtained by the incorporation of labeled ribonucleotides/deoxynucleotides during the amplification step in order to obtain target labeled polynucleotides according to the invention. Fluorescent labeled nucleotides are preferred since they are incorporated by the polymerase and lead to the formation of fluorescent labeled target polynucleotides. Cy3, Cy5 or Cy7 labeled nucleotides are preferred fluorochromes.

In another embodiment, the detection of the miRNA is performed after ligation of the miRNA hybridized on its complementary bait sequence with an adjacent probe.

In a preferred embodiment, the labeling of the miRNA is performed after ligation of the miRNA hybridized on its complementary bait sequence with an adjacent probe. Labeling may be obtained by using a labeled adjacent probe for ligation. In a preferred embodiment, the adjacent probe is pre-hybridized with its complementary bait sequence before ligation with the miRNA. Ligation is performed under conditions as to obtain formation of a DNA/DNA-RNA hybrid complex. Here, the DNA bait is used as a matrix and the DNA adjacent probe is utilized for ligation with miRNA for which the analysis is required. The DNA baits used for the formation of the hybrid complex is replaced by any nucleotide or nucleotide-like molecules as long as the ligation of the bound miRNA is possible. After denaturation, the labeled strand will be used for incubation with the capture probes present on the array for detection and quantification of the miRNA (FIG. 3). Preferably, the capture probes of the array are not complementary of the labeled adjacent probes in order to avoid false positive hybridizations.

According to a preferred embodiment, the ligation is performed with the T4 RNA ligase which ligates DNA sequences to RNA sequences such as miRNAs.

Detection of miRNA can be further enhanced by using a polynucleotide amplification step. This is accomplished using a mixture of DNA polymerase III or I of E. coli with a strand displacement DNA polymerase (ex. Bca DNApol I or phi29 DNApol) and circular DNA polynucleotide baits that are complementary to the sequence (miRNA or precursor) to be targeted. When the baits are annealing to their target sequences, a single strand concatenated polynucleotide is synthesized by the DNA polymerase. Labelled nucleotides are provided for incorporation during this amplification step. The resulting labelled single strand concatenated molecule is then hybridized on the microarray presenting complementary capture probes. The Single stranded DNA concatemer can be fragmented by hybridizing short oligonucleotides that reconstitute restriction sites. As an option, the concatenated molecule is fragmented by for ex. mild DNase treatment.

a) Preparation of the Circular Baits:

Two methods are preferred to prepare circular bait molecules in large scale.

1. They are produced by annealing the extremities of a linear single stranded bait DNA polynucleotide to a shorter (ex: 40-50 bases) single stranded polynucleotide. The overlapping sequence of both ends of the larger molecule is typically 25 bases and is complementary to the 50 bases polynucleotide. The annealed molecules are then treated by a DNA ligase specialised in ligation of single-stranded nicks in ds DNA molecules producing a circular bait polynucleotide. One preferred enzyme is the NAD⁺-dependent E. coli DNA ligase. The E. coli DNA ligase joins the 5′-end of the ss bait polynucleotide to its 3′-end when they are annealed next to each other on the shorter polynucleotide.

2. The ends of individual single-stranded bait molecules are ligated directly without preliminary hybridisation to a complementary short polynucleotide. This reaction is catalysed by a ssDNA Ligase specialised on intramolecular ligation of single-stranded DNA polynucleotides (CircLigase™ ssDNA Ligase, Epicentre Technologies, CL4111K).

In both cases, after the ligation the excess of the shorter (50 bases) molecule (annealed or free in solution) as well as non-ligated linear bait molecules are then removed with an exonuclease. A number exonuclease enzymes are preferably used for that purpose, comprising but not limited to exonuclease I, mung bean exonuclease, bacterial DNA polymerase III epsilon subunits or DNA polymerases with a strong 3′-5′ proof-reading exonuclease activity. After the incubation the exonucleases are inactivated by a heat treatment at 90-95° C.

b) miRNA Annealing in Solution and Detection of the Amplified Product on Microarray.

A pool of single stranded circular baits (as prepared in step a) targeting one or more miRNA's and precursor RNA molecules are hybridized in solution to the miRNA sample preparation. The annealed miRNAs then act as RNA primers for selected DNA-dependent DNA polymerases, preferably but not limited to the alpha subunits of bacterial DNA polymerases III or E. coli DNA polymerase I, to initiate DNA synthesis on the miRNA-primed bait template molecule. The RNA-primed bait-DNA polymerase reaction is further subjected to a second DNA polymerase with strong strand displacement activity, such as Bca DNA Pol I, Bst DNA Pol I or phi29 DNA polymerase, to transform the initial primer extension reaction into a rolling circle amplification synthesis (RCA). After rolling circle amplification and labelling, the polynucleotides are detected on a microarray presenting capture probes complementary to the amplified product (natural sequences or tags included in the bait). Optionally, the long single-stranded DNA molecules comprising DNA concatemers of miRNA sequences can be fragmented to miDNA monomers to facilitate hybridisation with capture probes in the array. The fragmentation is achieved in a sequence-specific manner by hybridisation to nonamer DNA-oligonucleotides, which are complementary to a unique restriction endonuclease site placed downstream of the miRNA sequence on the bait DNA, followed by incubation with the corresponding restriction endonuclease.

c) miRNA Annealing in Solid Phase and on Site Accumulation of the Amplification Products on the Microarray Without Hybridization Step.

The single stranded circular baits are immobilized on discrete regions at the surface of a substrate compatible with rolling circle amplification. The synthesis products, preferably laleled, then accumulated on site (on spot). In this case the circular RCA template (bait DNA) is surface-immobilized, whereas the other reactants (e.g. DNA polymerases, labeled and non-labeled dNTPs) are free in solution that covers the array surface.

In a preferred embodiment, elongation of the miRNAs is effected on complementary bait sequences being circular and single stranded. In an embodiment, the elongated miRNAs are amplified by rolling circle.

In another preferred embodiment, the bait sequences being circular and single stranded are capture probes arranged in specific locations of an array.

The present invention is also particularly suitable to detect and/or quantify the processed miRNA but also their precursors preferably the Pre-miRNA. The detection of precusrsor miRNA transcripts is achieved by using for each miRNA particular capture probes on the microarray that will be complementaty to some parts of the Pre-miRNA but located outside the 20-25 nt bound to the RISC and having no effect on the transcription, preferably sequences present in the loop.

In a preferred embodiment, the capture probes of the array are able to detect both precursor and mature miRNA forms. Simultaneous detection of the precursor pool and the processed or mature form of miRNA in a cell allows a more detailed understanding of the regulatory state of the cell for transcription.

In an embodiment, the capture probes contain LNA (locked nucleic acid) nucleotides. The detection of miRNA can be enhanced by using a capture probe with LNA nucleotide in the positions of the mismatches of the miRNA duplex.

In a next step (step (iii)), the miRNA or molecule derived therefrom (e.g. a DNA-copy or amplicon), is contacted with the array under conditions, allowing hybridization of the miRNA, or the molecule derived therefrom, with the capture probes present on the array. After a time sufficient for forming the duplex, a signal or a change in signal is detected on a specific location on the array.

In case the miRNA, or molecule derived therefrom, has been labelled prior to the hybridization step, the presence of fixed labelled target will be indicative of the presence of miRNA in the sample and, in knowledge of the gene to which it binds, also which transcript is controlled in the cell via this mechanism. The amount of fixed labelled target on the array will be proportional to the miRNA if performed under the appropriate conditions.

The presence of target bound on the different capture probes present on the solid support may be analyzed, identified and/or quantified by an apparatus comprising a detection and/or quantification device of a signal formed at the location of the binding between the target molecule and the capture molecule, preferably also a reading device of information recorded on a surface of said solid support, a computer program for recognizing the discrete regions bearing the bound target molecules upon its corresponding capture molecules and their locations, preferably also a quantification program of the signal present at the locations and a program for correlating the presence of the signal at these locations with the diagnostic and/or the quantification of the components to be detected according to the invention.

The principle laid down in the present specification may also be used in a method for determining the exact location of the miRNA binding on a gene sequence and/or the transcript. To this end, sequences of the gene or transcript, respectively, are arranged on the array on different locations, and upon hybridization it may be determined, to which part of the gene and/or transcript the miRNA binds.

In a preferred embodiment, the signal present on a specific location on the array corresponds to a pattern of at least 5, 10, 15, 20, 25, 30 and even 50 miRNAs.

In a preferred embodiemnt, the signal associated with a capture moleclue on the array is quantified. The preferred method is the scanning of the arrays with a scanner being preferentially a laser confocal scanner such as “ScanArray” (Packard, USA). The resolution of the image is comprised between 1 and 500 μm and preferably between 5 and 50 μm. To Preferably the arrays is scanned at different photomultiplier tube (PMT) settings in order to maximize the dynamic range and the data processed for quantification and corrections with the appropriated controls and standards (de Longueville et al, Biochem Pharmacol. 64, 2002, 137-49).

The knowledge provided by the present invention allows the design of new medicaments comprising sequences containing the RNAi sequences.

Also, the present invention is suitable for screening for compounds appropriate for regulation of gene translation or to follow cell reactions in the presence of biological or chemical compounds.

According to one embodiment, the cells, tissues or organisms are placed in the presence of one molecule and the analysis according to the present invention is carried out. The analysis of the spots intensities specific of the different genes gives an estimation and possible quantification of the miRNA present within the cells compared to cells incubated without the given compound. The invention is particularly useful for the determination of the efficiency of the transfection of the miRNA directed against one or several particular genes.

Variation in the level of the miRNA for particular genes are determined and give a first overview of the changes occurring in the biological organisms, cells or tissues, due to the compound. Compounds comprise: biological molecules such as cytokines, growth hormones, or any biological molecules affecting cells. Is also comprises chemical compounds such as drugs, toxic molecules, compounds from plants or animal extracts, chemicals resulting from organic synthesis including combinatory chemistry. The invention is particularly well suited for the screening of these compounds on cell regulation of the transcription of the genes. The overview of the changes in biological organisms is best obtained by screening for potentially active miRNA directed against the main vital cellular functions as following: apoptosis, cell adhesion, cell cycle, growth factors and cytokines, cell signaling, chromosomal processing, DNA repair/synthesis, intermediate metabolism, extracellular matrix, cell structure, protein metabolism, oxidative metabolism, transcription and house keeping genes. The invention best application is for the detection of miRNA against genes corresponding for the proteins involved in at least 9 of the 13 main cellular functions. In another embodiment, the array is used for the identification and/or quantification of miRNA present in cells against gene corresponding to at least 5 genes from one cellular functions including the 13 vital functions described above, but also including specialized functions such as cell differentiation, oncogene/tumor suppressor, stress response, lipid metabolism, proteasome, circulation. Also the invention is best when focused on genes related to one particular function which has biological, pharmaceutical, therapeutical or pathological interest.

In a particular embodiment, the detection and/or quantification of the gene expression is perfomed on the same sample as the detection and/or the quantification of the miRNA. Preferably the gene expression of at least 10 and preferably at least 50 genes is determined. The level of expression is then correlated with the presence and/or the amount of the miRNA assayed in the same sample preferably with the genes regulated or targeted by the assayed miRNA.

In one embodiment, cells, tissues or organisms are incubated in particular physical, chemical or biological conditions and the analysis performed according to the invention. Particular physical condition means only condition in which a physical parameter has been changed such as pH, temperature, pressure.

The particular chemical conditions mean any conditions in which the concentrations of one or several chemicals have been changed as compared to a control or reference condition including salts, oxygen, nutriments, proteins, glucides (carbohydrates), and lipids.

The particular biological conditions mean any changes in the living cells, tissues or organisms including ageing, stress, transformation (cancer), pathology, which affect cells, tissues or organisms.

Therefore, the method and support as described herein may be utilized as part of a diagnostic and/or quantification kit which comprises means and media for analyzing biological samples containing target molecules being detected after their binding onto the capture probes being present on the support in the form of array with a density of at least 4 different capture probes per cm.sup.2 of surface of rigid support. In its simple specification, the kit may also contain a support with a single capture probe.

Also provided by the present invention is a kit for the determination of miRNA mediated cellular transcriptional regulation in a sample comprising an array comprising at least 3 and preferably 20 and still preferably 50 capture probes being arranged in specific locations and optionally, buffers and labels. Preferably the array, harboring capture probes having at least part of their sequence identical or complementary to at least 3 and preferably 20 miRNA sequences provided in table 1 and/or 2 and/or 3 and being present at specific locations of the array, and buffers and labels. Also preferably the capture probes have a spacer being preferably located at a distance of 6.8 nm from the support and even preferably being a sequence of nucleotides being at least 20 bases and preferably more than 90 bases. The inventors found unexpected effect of the spacer leading to a large increase in the sensitivity of the detection on the array. The sensitivity is a particular issue of miRNA detection since they are present in cells at very low concentration and they must be detected in a very complex medium.

In still another embodiement the kit contains tools and reagent for the determination of miRNA mediated cellular transcriptional regulation in a sample and comprises two arrays comprising at least 3 capture probes being arranged in specific locations and reflecting the genomic or transcriptional matter of a cell, wherein the first array is dedicated to the detection and of multiple miRNAs present and the second array is dedicated to the detection and quantification of the expression of the regulated genes in the same sample and optionally, buffers and labels. The arrays are either present on the same supports or on different supports.

Also provided is a kit or a screening device for testing the effects of a compound on gene expression by detection of the presence of miRNA directed against at least one gene, said screening device comprising an array including capture probes having a sequence at least 90% homologous to mRNA or part thereof and being present at specific locations of the array and optionally buffers and labels.

It will be readily apparent to one of ordinary skill in the relevant arts that other suitable modifications and adaptations to the methods and applications described herein can be made without departing from the scope of the invention or any embodiment thereof. The present invention is described further by reference to the following example, which is illustrative only.

### EXAMPLE

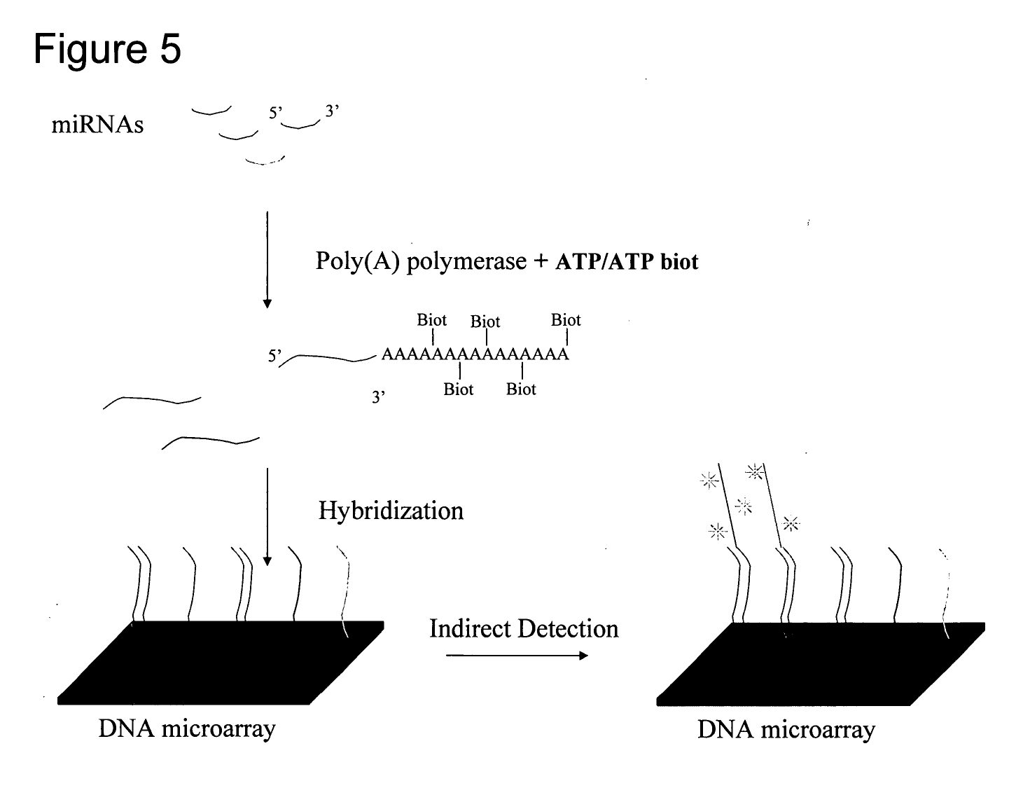

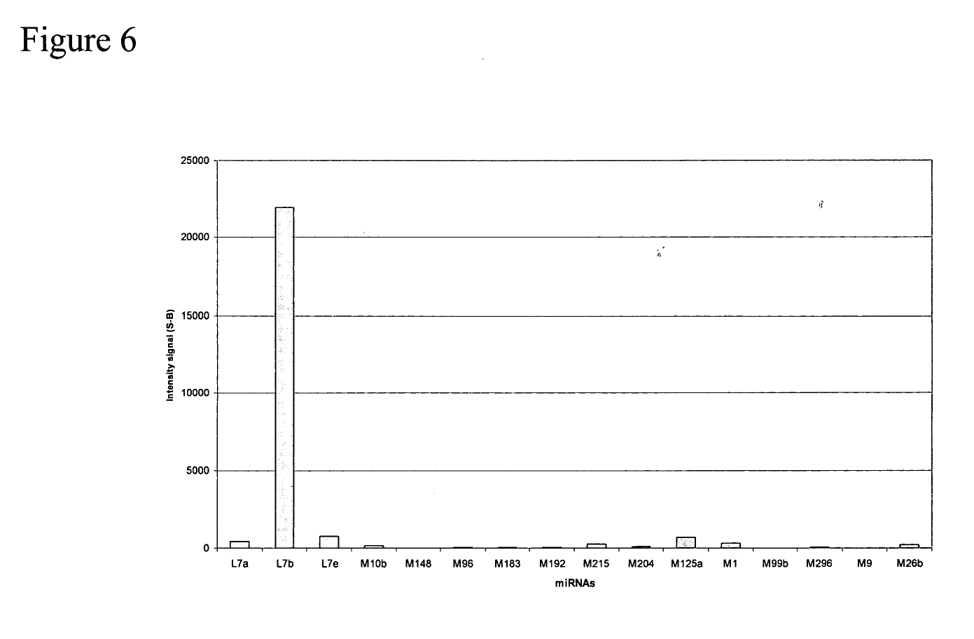

The experiment is performed as schematically described in FIG. 5 and the data are presented in the FIG. 6.

miRNA Extraction:

miRNAs are extracted from human brain tissue using the mirVana miRNA isolation procedure variant for isolation of RNA that is highly enriched for small RNAs (Ambion). The sample was disrupted in a denaturing lysis buffer and subsequently extracted in Acid-Phenol:Chloroform (Chomczynski and Sacchi, Anal. Biochem. 162 (1987), 156-159) ⅓ volume of 100% ethanol is added to the aqueous phase recovered from the organic extraction, mixed and passed through a glass filter cartridge (using centrifugal force). After this step, the filtrate was further enriched by adding ⅔ volume of 100% ethanol, mixed and applied on a second glass filter cartridge. The small RNA molecules remain trapped on the glass filter and are washed three times with a 45% ethanol solution. The RNA is then eluted with nuclease-free water and recovered in a collection tube.

miRNA Labelling:

The small size RNA population is then tailed and labelled with a mixture of biotinylated ATP and ATP using Poly(A) Polymerase enzyme (PAP) at the 3′ end of each miRNA. miRNA strand extension is performed with PAP (Ambion) for 60 min at 37° C. The labelled miRNA were then clean up (NucAway from Ambion).

Hybridization

The resulting product is then hybridized on the DualChips miRNA micro-array bearing ssDNA capture probes specific for mature miRNA sequences (Eppendorf, Hamburg, Germany).

The features of the capture probe are presented in the table below.

| | | | |
|---|---|---|---|
| Name | Sequence | Length | Tm |

| | | | |
|---|---|---|---|
| Let-7a | AACTATACAACCTACTACCTCA | 22 | 60° C. |
| Let-7b | AACCACACAACCTACTACCTCA | 22 | 64° C. |
| Let-7e | ACTATACAACCTCCTACCTCA | 21 | 60° C. |
| Mir-10b | ACAAATTCGGTTCTACAGGGTA | 22 | 62° C. |
| Mir-148a | ACAAAGTTCTGTAGTGCACTGA | 22 | 62° C. |
| Mir-96 | GCAAAAATGTGCTAGTGCCAAA | 22 | 62° C. |
| Mir-183 | CAGTGAATTCTACCAGTGCCATA | 23 | 66° C. |
| Mir-192 | GGCTGTCAATTCATAGGTCAG | 21 | 62° C. |
| Mir-215 | GTCTGTCAATTCATAGGTCAT | 21 | 58° C. |
| Mir-204 | AGGCATAGGATGACAAAGGGAA | 22 | 64° C. |
| Mir-125a | CACAGGTTAAAGGGTCTCAGGGA | 23 | 70° C. |
| Mir-1 | TACATACTTCTTTACATTCCA | 21 | 54° C. |
| Mir-99b | CGCAAGGTCGGTTCTACGGGTG | 22 | 72° C. |
| Mir-296 | ACAGGATTGAGGGGGGGCCCT | 21 | 70° C. |
| Mir-9 | ACTTTCGGTTATCTAGCTTTA | 21 | 56° C. |
| Mir-26b | ACCTATCCTGAATTACTTGAA | 21 | 56° C. |

Hybridization mixture consisted in biotinylated miRNA-DNA hybrid, 10 μl HybriBuffer A (Eppendorf, Hambourg, Germany), 40 μl HybriBuffer B (Eppendorf, Hambourg, Germany), 22 μl H2O, and 10 μl of positive hybridization control. Hybridization was carried out overnight at 60° C. The micro-arrays were then washed 4 times for 2 min with washing buffer (B1 0.1×+Tween 0.1%) (Eppendorf, Hamburg, Germany).

The micro-arrays were than incubated for 45 min at room temperature with the Cy3-conjugated IgG Anti biotin (Jackson Immuno Research laboratories, Inc #200-162-096) diluted 1/1000× Conjugate-Cy3 in the blocking reagent and protect from light. The micro-arrays were washed again 5 times for 2 min with washing buffer (B1 0.1×+Tween 0.1%) and 2 times for 2 min with distilled water before being dried under a flux of N2.

After image acquisition, the scanned 16-bit images are imported to the software, ‘ImaGene4.0’ (BioDiscovery, Los Angeles, Calif., USA), which is used to quantify the signal intensities. The spots intensities are first corrected by a subtraction of the local background intensity from signal intensity.

In order to evaluate the entire experiment, several positive and negative controls (for hybridization and detection) are first analysed. Then the signal obtained on each miRNA spots is analysed in order to correlate the result with the presence or not of miRNA directed against the specific gene in the sample.

The result of the FIG. 6 shows that the miRNA let 7b is highly expressed in brain tissue.

TABLE 1
miRNA human sequences

| | | | | | |
|---|---|---|---|---|---|
| ID | Species | Gene | miRNA sequence | Mature | Precursor |

| | | | | | |
|---|---|---|---|---|---|
| hsa-mir-7-1 | Homo sapiens | miR-7-1 | uggaagacuagugauuuuguu | 21 | 110 |
| hsa-mir-7-2 | Homo sapiens | miR-7-2 | uggaagacuagugauuuuguu | 21 | 110 |
| hsa-mir-7-3 | Homo sapiens | miR-7-3 | uggaagacuagugauuuuguu | 21 | 110 |
| hsa-let-7f-2L | Homo sapiens | let-7f-2 | ugagguaguagauuguauaguu | 22 | 89 |
| hsa-let-7f-1L | Homo sapiens | let-7f-1 | ugagguaguagauuguauaguu | 22 | 87 |
| hsa-let-7eL | Homo sapiens | let-7e | ugagguaggagguuguauagu | 21 | 79 |
| hsa-let-7a-1L | Homo sapiens | let-7a-1 | ugagguaguagguuguauaguu | 22 | 80 |
| hsa-let-7a-2L | Homo sapiens | let-7a-2 | ugagguaguagguuguauaguu | 22 | 72 |
| hsa-Iet-7a-3L | Homo sapiens | let-7a-3 | ugagguaguagguuguauaguu | 22 | 74 |
| hsa-let-7bL | Homo sapiens | let-7b | ugagguaguagguugugugguu | 22 | 83 |
| hsa-let-7cL | Homo sapiens | let-7c | ugagguaguagguuguaugguu | 22 | 84 |
| hsa-let-7dL | Homo sapiens | let-7d | agagguaguagguugcauagu | 21 | 87 |
| hsa-mir-10a | Homo sapiens | mir-10a | uacccuguagauccgaauuugug | 23 | 110 |
| hsa-mir-10b | Homo sapiens | mir-10b | uacccuguagaaccgaauuugu | 22 | 110 |
| hsa-mir-15 | Homo sapiens | mir-15 | uagcagcacauaaugguuugug | 22 | 83 |
| hsa-mir-16 | Homo sapiens | mir-16 | uagcagcacguaaauauuggcg | 22 | 89 |
| hsa-mir-17 | Homo sapiens | mir-17 | acugcagugaaggcacuugu | 20 | 84 |
| hsa-mir-18 | Homo sapiens | mir-18 | uaaggugcaucuagugcagaua | 22 | 71 |
| hsa-mir-19a | Homo sapiens | mir-19 | augugcaaaucuaugcaaaacuga | 23 | 82 |
| hsa-mir-19b-1 | Homo sapiens | mir-19b-1 | ugugcaaauccaugcaaaacuga | 23 | 87 |
| hsa-mir-19b-2 | Homo sapiens | mir-19b-2 | ugugcaaauccaugcaaaacuga | 23 | 96 |
| hsa-mir-20 | Homo sapiens | mir-20 | uaaagugcuuauagugcaggua | 22 | 71 |
| hsa-mir-21 | Homo sapiens | mir-21 | uagcuuaucagacugauguuga | 22 | 72 |
| hsa-mir-22 | Homo sapiens | mir-22 | aagcugccaguugaagaacugu | 22 | 85 |
| hsa-mir-23 | Homo sapiens | mir-23 | aucacauugccagggauuucc | 21 | 73 |
| hsa-mir-24-2 | Homo sapiens | mir-24-2 | uggcucaguucagcaggaacag | 22 | 73 |
| hsa-mir-24-1 | Homo sapiens | mir-24-1 | uggcucaguucagcaggaacag | 22 | 68 |
| hsa-mir-25 | Homo sapiens | mir-25 | cauugcacuugucucggucuga | 22 | 84 |
| hsa-mir-26a | Homo sapiens | mir-26a | uucaaguaauccaggauaggcu | 22 | 75 |
| hsa-mir-26b | Homo sapiens | mir-26b | uucaaguaauucaggauaggu | 21 | 77 |
| hsa-mir-27 | Homo sapiens | mir-27 | uucacaguggcuaaguuccgcc | 22 | 78 |
| hsa-mir-28 | Homo sapiens | mir-28 | aaggagcucacagucuauugag | 22 | 86 |
| hsa-mir-29 | Homo sapiens | mir-29 | cuagcaccaucugaaaucgguu | 22 | 64 |
| hsa-mir-30c | Homo sapiens | mir-30c | uguaaacauccuacacucucagc | 23 | 72 |
| hsa-mir-30d | Homo sapiens | mir-30d | uguaaacauccccgacuggaag | 22 | 70 |
| hsa-mir-30a | Homo sapiens | mir-30a-s | uguaaacauccucgacuggaagc | 23 | 71 |

The mature sequences miR-30 and miR-97 appear to originate from the same pre-
cursor and the entries have been merged and renamed to match the homologous
mouse entry.

| | | | | | |
|---|---|---|---|---|---|
| hsa-mir-30a | Homo sapiens | mir-30a-as | cuuucagucggauguuugcagc | 22 | 71 |
| hsa-mir-31 | Homo sapiens | mir-31 | ggcaagaugcuggcauagcug | 21 | 71 |
| hsa-mir-32 | Homo sapiens | mir-32 | uauugcacauuacuaaguugc | 21 | 70 |
| hsa-mir-33 | Homo sapiens | mir-33 | gugcauuguaguugcauug | 19 | 69 |
| hsa-mir-34 | Homo sapiens | mir-34 | uggcagugucuuagcugguugu | 22 | 110 |
| hsa-mir-91 | Homo sapiens | mir-91 | caaagugcuuacagugcagguagu | 24 | 82 |
| — | Homo sapiens | mir-17 | acugcagugaaggcacuugu | 20 | 82 |

miR-17 is cleaved from the reverse strand of human precursor mir-91 and from
human precursor mir-17

| | | | | | |
|---|---|---|---|---|---|
| hsa-mir-92-1 | Homo sapiens | mir-92-1 | uauugcacuugucccggccugu | 22 | 78 |
| hsa-mir-92-2 | Homo sapiens | mir-92-2 | uauugcacuugucccggccugu | 22 | 75 |
| hsa-mir-93-1 | Homo sapiens | mir-93-1 | aaagugcuguucgugcagguag | 22 | 80 |
| hsa-mir-93-2 | Homo sapiens | mir-93-2 | aaagugcuguucgugcagguag | 22 | 80 |
| hsa-mir-95 | Homo sapiens | mir-95 | uucaacggguauuuauugagca | 22 | 81 |
| hsa-mir-96 | Homo sapiens | mir-96 | uuuggcacuagcacauuuuugc | 22 | 78 |
| hsa-mir-98 | Homo sapiens | mir-98 | ugagguaguaaguuguauuguu | 22 | 80 |
| hsa-mir-99 | Homo sapiens | mir-99 | aacccguagauccgaucuugug | 22 | 81 |
| hsa-mir-100 | Homo sapiens | mir-100 | aacccguagauccgaacuugug | 22 | 80 |
| hsa-mir-101 | Homo sapiens | mir-101 | uacaguacugugauaacugaag | 22 | 75 |
| hsa-mir-102-1 | Homo sapiens | mir-102-1 | uagcaccauuugaaaucagu | 20 | 81 |
| hsa-mir-102-2 | Homo sapiens | mir-102-2 | uagcaccauuugaaaucagu | 20 | 81 |
| hsa-mir-102-3 | Homo sapiens | mir-102-3 | uagcaccauuugaaucagu | 20 | 81 |
| hsa-mir-103-2 | Homo sapiens | mir-103-2 | agcaacauuguacagggcuauga | 23 | 78 |
| hsa-mir-103-1 | Homo sapiens | mir-103-1 | agcagcauuguacagggcuauga | 23 | 78 |
| hsa-mir-104 | Homo sapiens | mir-104 | ucaacaucagucugauaagcua | 22 | 78 |
| hsa-mir-105-1 | Homo sapiens | mir-105-1 | ucaaaugcucagacuccugu | 20 | 81 |
| hsa-mir-105-2 | Homo sapiens | mir-105-2 | ucaaaugcucagacuccugu | 20 | 81 |
| hsa-mir-106 | Homo sapiens | mir-106 | aaaagugcuuacagugcagguagc | 24 | 81 |
| hsa-mir-107 | Homo sapiens | mir-107 | agcagcauuguacagggcuauca | 23 | 81 |
| hsa-mir-124b | Homo sapiens | mir-124b | uuaaggcacgcggugaaugc | 20 | 67 |
| hsa-mir-139 | Homo sapiens | mir-139 | ucuacagugcacgugucu | 18 | 68 |
| hsa-mir-147 | Homo sapiens | mir-147 | guguguggaaaugcuucugc | 20 | 72 |
| hsa-mir-148 | Homo sapiens | mir-148 | ucagugcacuacagaacuuugu | 22 | 68 |
| hsa-mir-181c | Homo sapiens | mir-181c | aacauucaaccugucggugagu | 22 | 110 |
| hsa-mir-181b | Homo sapiens | mir-181b | accaucgaccguugauuguacc | 22 | 110 |
| hsa-mir-181a | Homo sapiens | mir-181a | aacauucaacgcugucggugagu | 23 | 110 |
| hsa-mir-182-as | Homo sapiens | mir-182-as | ugguucuagacuugccaacua | 21 | 110 |
| hsa-mir-183 | Homo sapiens | mir-183 | uauggcacugguagaauucacug | 23 | 110 |
| hsa-mir-187 | Homo sapiens | mir-187 | ucgugucuuguguugcagccg | 21 | 110 |
| hsa-mir-192 | Homo sapiens | mir-192 | cugaccuaugaauugacagcc | 21 | 110 |
| hsa-mir-196-2 | Homo sapiens | mir-196-2 | uagguaguuucauguuguuggg | 22 | 110 |
| hsa-mir-196-1 | Homo sapiens | mir-196-1 | uagguaguuucauguuguuggg | 22 | 110 |
| hsa-mir-196 | Homo sapiens | mir-196 | uagguaguuucauguuguugg | 21 | 70 |
| hsa-mir-197 | Homo sapiens | mir-197 | uucaccaccuucuccacccagc | 22 | 75 |
| hsa-mir-198 | Homo sapiens | mir-198 | gguccagaggggagauagg | 19 | 62 |
| hsa-mir-199a-2 | Homo sapiens | mir-199a-2 | cccaguguucagacuaccuguuc | 23 | 110 |
| hsa-mir-199b | Homo sapiens | mir-199b | cccaguguuuagacuaucuguuc | 23 | 110 |
| hsa-mir-199a-1 | Homo sapiens | mir-199a-1 | cccaguguucagacuaccuguuc | 23 | 110 |
| hsa-mir-199-s | Homo sapiens | mir-199-s | cccaguguucagacuaccuguu | 22 | 71 |
| hsa-mir-200b | Homo sapiens | mir-200b | cucuaauacugccugguaaugaug | 24 | 95 |
| hsa-mir-203 | Homo sapiens | mir-203 | gugaaauguuuaggaccacuag | 22 | 110 |
| hsa-mir-204 | Homo sapiens | mir-204 | uucccuuugucauccuaugccu | 22 | 110 |
| hsa-mir-205 | Homo sapiens | mir-205 | uccuucauuccaccggagucug | 22 | 110 |
| hsa-mir-208 | Homo sapiens | mir-208 | auaagacgagcaaaaagcuugu | 22 | 71 |
| hsa-mir-210 | Homo sapiens | mir-210 | cugugcgugugacagcggcug | 21 | 110 |
| hsa-mir-211 | Homo sapiens | mir-211 | uucccuuugucauccuucgccu | 22 | 110 |
| hsa-mir-212 | Homo sapiens | mir-212 | uaacagucuccagucacggcc | 21 | 110 |
| hsa-mir-213 | Homo sapiens | mir-213 | aacauucauugcugucgguggguu | 24 | 110 |
| hsa-mir-214 | Homo sapiens | mir-214 | acagcaggcacagacaggcag | 21 | 110 |
| hsa-mir-215 | Homo sapiens | mir-215 | augaccuaugaauugacagac | 21 | 110 |
| hsa-mir-216 | Homo sapiens | mir-216 | uaaucucagcuggcaacugug | 21 | 110 |
| hsa-mir-217 | Homo sapiens | mir-217 | uacugcaucaggaacugauuggau | 24 | 110 |
| hsa-mir-218-1 | Homo sapiens | mir-218-1 | uugugcuugaucuaaccaugu | 21 | 110 |
| hsa-mir-218-2 | Homo sapiens | mir-218-2 | uugugcuugaucuaaccaugu | 21 | 110 |
| hsa-mir-219 | Homo sapiens | mir-219 | ugauuguccaaacgcaauucu | 21 | 110 |
| hsa-mir-220 | Homo sapiens | mir-220 | ccacaccguaucugacacuuu | 21 | 110 |
| hsa-mir-221 | Homo sapiens | mir-221 | agcuacauugucugcuggguuuc | 23 | 110 |
| hsa-mir-222 | Homo sapiens | mir-222 | agcuacaucuggcuacugggucuc | 24 | 110 |
| hsa-mir-223 | Homo sapiens | mir-223 | ugucaguuugucaaauacccc | 21 | 110 |
| hsa-mir-224 | Homo sapiens | mir-224 | caagucacuagugguuccguuua | 23 | 81 |

TABLE 2
miRNA mouse sequences

| | | | | |
|---|---|---|---|---|
| ID | Species | Gene | mirNA sequence | Mature |

| | | | | |
|---|---|---|---|---|
| mmu-mir-1b | Mus musculus | mir-1b | UGGAAUGUAAAGAAGUAUGUAA | 22 |
| mmu-mir-1c | Mus musculus | mir-1c | UGGAAUGUAAAGAAGUAUGUAC | 22 |
| mmu-mir-1d | Mus musculus | mir-1d | UGGAAUGUAAAGAAGUAUGUAUU | 23 |
| mmu-mir-9 | Mus musculus | mir-9 | UCUUUGGUUAUCUAGCUGUAUGA | 23 |
| mmu-mir-9-star | Mus musculus | mir-9-star | UAAAGCUAGAUAACCGAAAGU | 21 |
| mmu-mir-10b | Mus musculus | mir-10b | CCCUGUAGAACCGAAUUUGUGU | 22 |
| mmu-mir-15a | Mus musculus | mir-15a | UAGCAGCACAUAAUGGUUUGUG | 22 |
| mmu-mir-15b | Mus musculus | mir-15b | UAGCAGCACAUCAUGGUUUACA | 22 |
| mmu-mir-16 | Mus musculus | mir-16 | UAGCAGCACGUAAAUAUUGGCG | 22 |
| mmu-mir-18 | Mus musculus | mir-18 | UAAGGUGCAUCUAGUGCAGAUA | 22 |
| mmu-mir-19b | Mus musculus | mir-19b | UGUGCAAAUCCAUGCAAAACUGA | 23 |
| mmu-mir-20 | Mus musculus | mir-20 | UAAAGUGCUUAUAGUGCAGGUAG | 23 |
| mmu-mir-21 | Mus musculus | mir-21 | UAGCUUAUCAGACUGAUGUUGA | 22 |
| mmu-mir-22 | Mus musculus | mir-22 | AAGCUGCCAGUUGAAGAACUGU | 22 |
| mmu-mir-23a | Mus musculus | mir-23a | AUCACAUUGCCAGGGAUUUCC | 21 |
| mmu-mir-23b | Mus musculus | mir-23b | AUCACAUUGCCAGGGAUUACCAC | 23 |
| mmu-mir-24 | Mus musculus | mir-24 | UGGCUCAGUUCAGCAGGAACAG | 22 |
| mmu-mir-26a | Mus musculus | mir-26a | UUCAAGUAAUCCAGGAUAGGCU | 22 |
| mmu-mir-26b | Mus musculus | mir-26b | UUCAAGUAAUUCAGGAUAGGUU | 22 |
| mmu-mir-27a | Mus musculus | mir-27a | UUCACAGUGGCUAAGUUCCGCU | 22 |
| mmu-mir-27b | Mus musculus | mir-27b | UUCACAGUGGCUAAGUUCUG | 20 |
| mmu-mir-29a | Mus musculus | mir-29a | CUAGCACCAUCUGAAAUCGGUU | 22 |
| mmu-mir-29b | Mus musculus | mir-29b | UAGCACCAUUUGAAAUCAGUGUU | 23 |
| mmu-mir-29c | Mus musculus | mir-29c | UAGCACCAUUUGAAAUCGGUUA | 22 |
| mmu-mir-30a | Mus musculus | mir-30a | UGUAAACAUCCUCGACUGGAAGC | 23 |
| mmu-mir-30a-as | Mus musculus | mir-30a-as | CUUUCAGUCGGAUGUUUGCAGC | 22 |
| mmu-mir-30bb | Mus musculus | mir-30b | UGUAAACAUCCUACACUCAGC | 21 |
| mmu-mir-30c | Mus musculus | mir-30c | UGUAAACAUCCUACACUCUCAGC | 23 |
| mmu-mir-30d | Mus musculus | mir-30d | UGUAAACAUCCCCGACUGGAAG | 22 |
| mmu-mir-99a | Mus musculus | mir-99a | ACCCGUAGAUCCGAUCUUGU | 20 |
| mmu-mir-99b | Mus musculus | mir-99b | CACCCGUAGAACCGACCUUGCG | 22 |
| mmu-mir-101 | Mus musculus | mir-101 | UACAGUACUGUGAUAACUGA | 20 |
| mmu-mir-122a | Mus musculus | mir-122a | UGGAGUGUGACAAUGGUGUUUGU | 23 |
| mmu-mir-122b | Mus musculus | mir-122b | UGGAGUGUGACAAUGGUGUUUGA | 23 |
| mmu-mir-124a | Mus musculus | mir-124a | UUAAGGCACGCGGUGAAUGCCA | 22 |
| mmu-mir-124b | Mus musculus | mir-124b | UUAAGGCACGCGGGUGAAUGC | 21 |
| mmu-mir-125a | Mus musculus | mir-125a | UCCCUGAGACCCUUUAACCUGUG | 23 |
| mmu-mir-125b | Mus musculus | mir-125b | UCCCUGAGACCCUAACUUGUGA | 22 |
| mmu-mir-126 | Mus musculus | mir-126 | UCGUACCGUGAGUAAUAAUGC | 21 |
| mmu-mir-126-star | Mus musculus | mir-126-star | CAUUAUUACUUUUGGUACGCG | 21 |
| mmu-mir-127 | Mus musculus | mir-127 | UCGGAUCCGUCUGAGCUUGGCU | 22 |
| mmu-mir-128 | Mus musculus | mir-128 | UCACAGUGAACCGGUCUCUUUU | 22 |
| mmu-mir-129 | Mus musculus | mir-129 | CUUUUUUCGGUCUGGGCUUGC | 21 |
| mmu-mir-129b | Mus musculus | mir-129b | CUUUUUGCGGUCUGGGCUUGCU | 22 |
| mmu-mir-130 | Mus musculus | mir-130 | CAGUGCAAUGUUAAAAGGGC | 20 |
| mmu-mir-132 | Mus musculus | mir-132 | UAACAGUCUACAGCCAUGGUCGU | 23 |
| mmu-mir-133 | Mus musculus | mir-133 | UUGGUCCCCUUCAACCAGCUGU | 22 |
| mmu-mir-134 | Mus musculus | mir-134 | UGUGACUGGUUGACCAGAGGGA | 22 |
| mmu-mir-135 | Mus musculus | mir-135 | UAUGGCUUUUUAUUCCUAUGUGAA | 24 |
| mmu-mir-136 | Mus musculus | mir-136 | ACUCCAUUUGUUUUGAUGAUGGA | 23 |
| mmu-mir-137 | Mus musculus | mir-137 | UAUUGCUUAAGAAUACGCGUAG | 22 |
| mmu-mir-138 | Mus musculus | mir-138 | AGCUGGUGUUGUGAAUC | 17 |
| mmu-mir-139 | Mus musculus | mir-139 | UCUACAGUGCACGUGUCU | 18 |
| mmu-mir-140 | Mus musculus | mir-140 | AGUGGUUUUACCCUAUGGUAG | 21 |
| mmu-mir-141 | Mus musculus | mir-141 | AACACUGUCUGGUAAAGAUGG | 21 |
| mmu-mir-142s | Mus musculus | mir-142s | CAUAAAGUAGAAAGCACUAC | 20 |
| mmu-mir-142as | Mus musculus | mir-142as | UGUAGUGUUUCCUACUUUAUGG | 22 |
| mmu-mir-143 | Mus musculus | mir-143 | UGAGAUGAAGCACUGUAGCUCA | 22 |
| mmu-mir-144 | Mus musculus | mir-144 | UACAGUAUAGAUGAUGUACUAG | 22 |
| mmu-mir-145 | Mus musculus | mir-145 | GUCCAGUUUUCCCAGGAAUCCCUU | 24 |
| mmu-mir-146 | Mus musculus | mir-146 | UGAGAACUGAAUUCCAUGGGUUU | 23 |
| mmu-mir-147 | Mus musculus | mir-147 | GUGUGUGGAAAUGCUUCUGCC | 21 |
| mmu-mir-148 | Mus musculus | mir-148 | UCAGUGCACUACAGAACUUUGU | 22 |
| mmu-mir-149 | Mus musculus | mir-149 | UCUGGCUCCGUGUCUUCACUCC | 22 |
| mmu-mir-150 | Mus musculus | mir-150 | UCUCCCAACCCUUGUACCAGUGU | 23 |
| mmu-mir-151 | Mus musculus | mir-151 | CUAGACUGAGGCUCCUUGAGGU | 22 |
| mmu-mir-152 | Mus musculus | mir-152 | UCAGUGCAUGACAGAACUUGG | 21 |
| mmu-mir-153 | Mus musculus | mir-153 | UUGCAUAGUCACAAAAGUGA | 20 |
| mmu-mir-154 | Mus musculus | mir-154 | UAGGUUAUCCGUGUUGCCUUCG | 22 |
| mmu-mir-155 | Mus musculus | mir-155 | UUAAUGCUAAUUGUGAUAGGGG | 22 |
| mmu-mir-181 | Mus musculus | mir-181 | AACAUUCAACGCUGUCGGUGAGU | 23 |
| mmu-mir-182 | Mus musculus | mir-182 | UUUGGCAAUGGUAGAACUCACA | 22 |
| mmu-mir-183 | Mus musculus | mir-183 | UAUGGCACUGGUAGAAUUCACUG | 23 |
| mmu-mir-184 | Mus musculus | mir-184 | UGGACGGAGAACUGAUAAGGGU | 22 |
| mmu-mir-185 | Mus musculus | mir-185 | UGGAGAGAAAGGCAGUUC | 18 |
| mmu-mir-186 | Mus musculus | mir-186 | CAAAGAAUUCUCCUUUUGGGCUU | 23 |
| mmu-mir-187 | Mus musculus | mir-187 | UCGUGUCUUGUGUUGCAGCCGG | 22 |
| mmu-mir-188 | Mus musculus | mir-188 | CAUCCCUUGCAUGGUGGAGGGU | 22 |
| mmu-mir-189 | Mus musculus | mir-189 | GUGCCUACUGAGCUGACAUCAGU | 23 |
| mmu-mir-190 | Mus musculus | mir-190 | UGAUAUGUUUGAUAUAUUAGGU | 22 |
| mmu-mir-191 | Mus musculus | mir-191 | CAACGGAAUCCCAAAAGCAGCU | 22 |
| mmu-mir-192 | Mus musculus | mir-192 | CUGACCUAUGAAUUGACA | 18 |
| mmu-mir-193 | Mus musculus | mir-193 | AACUGGCCUACAAAGUCCCAG | 21 |
| mmu-mir-194 | Mus musculus | mir-194 | UGUAACAGCAACUCCAUGUGGA | 22 |
| mmu-mir-195 | Mus musculus | mir-195 | UAGCAGCACAGAAAUAUUGGC | 21 |
| mmu-mir-196 | Mus musculus | mir-196 | UAGGUAGUUUCAUGUUGUUGG | 21 |
| mmu-mir-199 | Mus musculus | mir-199s | CCCAGUGUUCAGACUACCUGUU | 22 |
| mmu-mir-199as | Mus musculus | mir-199as | UACAGUAGUCUGCACAUUGGUU | 22 |
| mmu-mir-200a | Mus musculus | mir-200a | UAACACUGUCUGGUAACGAUGU | 22 |
| mmu-mir-200b | Mus musculus | mir-200b | UAAUACUGCCUGGUAAUGAUGAC | 23 |
| mmu-mir-201 | Mus musculus | mir-201 | UACUCAGUAAGGCAUUGUUCU | 21 |
| mmu-mir-202 | Mus musculus | mir-202 | AGAGGUAUAGCGCAUGGGAAGA | 22 |
| mmu-mir-203 | Mus musculus | mir-203 | GUGAAAUGUUUAGGACCACUAGA | 23 |
| mmu-mir-204 | Mus musculus | mir-204 | UUCCCUUUGUCAUCCUAUGCCUG | 23 |
| mmu-mir-205 | Mus musculus | mir-205 | UCCUUCAUUCCACCGGAGUCUG | 22 |
| mmu-mir-206 | Mus musculus | mir-206 | UGGAAUGUAAGGAAGUGUGUGG | 22 |
| mmu-mir-207 | Mus musculus | mir-207 | GCUUCUCCUGGCUCUCCUCCCUC | 23 |
| mmu-mir-208 | Mus musculus | mir-208 | AUAAGACGAGCAAAAAGCUUGU | 22 |
| mmu-let-7a | Mus musculus | let-7a | UGAGGUAGUAGGUUGUGUGGUU | 22 |
| mmu-let-7b | Mus musculus | let-7b | UGAGGUAGUAGGUUGUAUAGUU | 22 |
| mmu-let-7c | Mus musculus | let-7c | UGAGGUAGUAGGUUGUAUGGUU | 22 |
| mmu-let-7d | Mus musculus | let-7d | AGAGGUAGUAGGUUGCAUAGU | 21 |
| mmu-let-7e | Mus musculus | let-7e | UGAGGUAGGAGGUUGUAUAGU | 21 |
| mmu-let-7f-1 | Mus musculus | let-7f-1 | UGAGGUAGUAGAUUGUAUAGUU | 22 |
| mmu-let-7f-2 | Mus musculus | let-7f-2 | UGAGGUAGUAGAUUGUAUAGUU | 22 |
| mmu-let-7g | Mus musculus | let-7g | UGAGGUAGUAGUUUGUACAGUA | 22 |
| mmu-let-7h | Mus musculus | let-7h | UGAGGUAGUAGUGUGUACAGUU | 22 |
| mmu-let-7i | Mus musculus | let-7i | UGAGGUAGUAGUUUGUGCU | 19 |

TABLE 3
Examples of miRNA human sequences and their targeted genes

| | | | | |
|---|---|---|---|---|
| Species | Gene | Sequence | Tissue's Localisation | Predicted Targeted genes |

| | | | | |
|---|---|---|---|---|
| Homo sapiens | let-7a | UGAGGUAGUAGGUUGUAUAGUU | Thymus | FSD1, MAP4K3, MAP3K1 |
| Homo sapiens | let-7b | UGAGGUAGUAGGUUGUGUGGUU | Brain | E2F5, CDH23, PCDH17 |
| Homo sapiens | let-7e | UGAGGUAGGAGGUUGUAUAGU | Testes | CCNL1, PDGFB, IMP3 |
| Homo sapiens | miR-10b | UACCCUGUAJAACCGAAUUUGU | Testes | MAP4, FBS1, RGL1 |
| Homo sapiens | miR-96 | UUUGGCACUAGCACAUUUUUGC | Thymus | TCF8, MRPL43, SLC20A1 |
| Homo sapiens | miR-148 | UCAGUGCACUACAGAACUUUGU | Liver | CDK5R1, PPARG, APOE |
| Homo sapiens | miR-183 | UAUGGCACUGGUAGAAUUCACUG | Thymus | MAP3K4, TNFSF11, DUSP10 |
| Homo sapiens | miR-192 | CUGACCUAUGAAUUGACAGCC | Kidney | HOXB2, UBE2D3, ZFHX4 |
| Homo sapiens | miR-204 | UUCCCUUUGUCAUCCUAUGCCU | Kidney | CREB5, BCL2, TFAP2C |
| Homo sapiens | miR-215 | AUGACCUAUGAAUUGACAGAC | Kidney | FGF10, TCF7L1, CIT |

We claim:

1. A method for detecting a miRNA directed against at least one specific gene present in a sample comprising the steps of: (i) isolating miRNA from a target cell; (ii) contacting the miRNA with an array of capture probes under hybridization conditions; and (iii) detecting a signal or a change in a signal on the array.

2. The method of claim 1, for determining the RNAi mediated transcriptional regulation in a cell by the determination of a pattern of miRNA detected simultaneously and quantified in the same cell extract, the method comprising the steps of:
(i) providing an array onto which at least 3 capture probes, are arranged in specific locations thereof;
(ii) isolating a miRNA pool potentially present from a cell;
(iii) elongating or ligating said miRNAs into target labeled polynucleotides;
(iv) contacting said target labeled polynucleotides with the array under conditions allowing hybridization of the target labeled polynucleotides to complementary capture probes present on the array;
(v) detecting and quantifying a signal present in specific locations on the array;
wherein the detection of a pattern of at least 3 signals on the array reflects the pattern of miRNAs being involved in the RNAi mediated cellular transcriptional regulation.

3. The method of claim 2, wherein the RNAi mediated cellular transcriptional regulation provided by the detection and quantification of a pattern of miRNAs is correlated with the pattern of expression of the regulated genes in the same sample.

4. The method of claim 2, wherein the RNAi mediated cellular transcriptional regulation provided by the detection and quantification of a pattern of miRNAs is correlated with the pattern of expression of the miRNA targeted genes in the same sample.

5. The method of claim 2, wherein the RNAi mediated cellular transcriptional regulation provided by the detection and quantification of a pattern of miRNAs is correlated with the pattern of expression of the genes having mRNA sequences having more than 90% homology to the corresponding miRNA sequences in the same sample.

6. The method of claim 2, wherein the RNAi mediated cellular transcriptional regulation is related to the development of an organism.

7. The method of claim 2, wherein the RNAi mediated cellular transcriptional regulation is related to cell differentiation or stem cell maintenance.

8. The method of claim 2 wherein the RNAi mediated cellular transcriptional regulation is related to cell proliferation.

9. The method of claim 2, wherein the RNAi mediated cellular transcriptional regulation is related to cell death.

10. The method of claim 2, wherein the RNAi mediated cellular transcriptional regulation is related to chromatin condensation.

11. The method of claim 2, wherein the RNAi mediated cellular transcriptional regulation is related to cell transformation.

12. The method of claim 1, wherein the miRNA is incorporated into a labeled DNA-RNA sequence which is then detected on the array.

13. The method of claim 2, wherein elongation of the miRNA hybridized on its complementary bait sequence is effected with the Tth DNA polymerase 3.

14. The method of claim 2, wherein elongation of the miRNA is performed by tailing the miRNA using the Poly A polymerase.

15. The method of claim 2, wherein ligation of the miRNA hybridized on its complementary bait sequence is effected by ligation with an adjacent probe.

16. The method of claim 15, wherein the adjacent probe is pre-hybridized with its complementary sequence before ligation with the miRNA.

17. The method of claim 15, wherein ligation of the miRNA with the adjacent probe is effected with the T4 RNA ligase.

18. The method of claim 15, wherein the adjacent probe is labeled.

19. The method of claim 2, wherein the elongation of the miRNA is effected on a sequence comprising three parts, the 3′ end is complementary of the miRNA, the middle part is specific of each bait and the 5′ end sequence is common to all baits.

20. The method of claim 19, wherein the elongated miRNAs are amplified.

21. The method of claim 20, wherein the amplification is performed after miRNA degradation using as matrix for the amplification a DNA/DNA hybrid complex.

22. The method of claim 19, wherein a primer complementary of the common sequence of the elongated DNA is provided for amplification.

23. The method of claim 22, wherein the amplification is performed with a DNA polymerase.

24. The method of claim 22, wherein the primer comprises a T7 promoter sequence for an RNA polymerase.

25. The method of claim 22, wherein the primer comprises a Tag sequence.

26. The method of claim 22, wherein the primer is used for in vitro transcription with a RNA polymerase.

27. The method of claim 1, wherein the array comprises capture probes ranging from 10 to about 1000 nucleotides, preferably from 15 to 200, or 15 to 100 nucleotides.

28. The method of claim 1, wherein the array comprises between 5-1000 and still preferably between 50-300 different capture probes.

29. The method of claim 1, wherein the signals present on the array correspond to a pattern of at least 10 miRNAs, preferably at least 20 miRNAs.

30. The method of claim 27, wherein the capture probes have sequences which are at least 90% homologous for at least 10 to 1000 nucleotides to same part of the mRNA corresponding to the miRNA to be detected.

31. The method of claim 1, wherein at least 3 and preferably 20 and more preferably 50 of the miRNA presented in Table 1 are simultaneously detected.

32. The method of claim 1, wherein at least 3 and preferably 20 and more preferably 50 of the miRNA presented in Table 2 are simultaneously detected.

33. The method of claim 1, wherein at least 3 and preferably 5 and more preferably 10 of the miRNA presented in Table 3 are simultaneously detected.

34. The method of claim 1, wherein the array comprises capture probes having at least part of their sequence being complementary of the miRNA and having between 15 and 25 bases and even preferably between 19 and 23 bases.

35. The method of claim 1, wherein the array comprises capture probes having specific sequences for the binding of the miRNA and a spacer being preferably located at a distance of 6.8 nm from the support and even preferably being a sequence of nucleotides being at least 20 bases and preferably more than 40 bases and even better 90 bases.

36. The method of claim 35, wherein the specific sequence of the capture probes has a Tm between 54 and 72° C. and preferably between 62 and 66° C.

37. The method of claim 1, wherein the capture probes are able to detect both precursor and mature miRNA forms.

38. The method of claim 2, wherein the elongation of the miRNAs is effected on complementary bait sequences being circular and single stranded.

39. The method of claim 38, wherein the elongated miRNAs are amplified by rolling circle.

40. The method of claim 38, wherein the bait sequences being circular and single stranded are capture probes arranged in specific locations of an array.

41. A kit for the determination of miRNA mediated cellular transcriptional regulation in a sample comprising an array comprising at least 3 and preferably 20 and still preferably 50 capture probes being arranged in specific locations and optionally, buffers and labels.

42. A kit of claim 41, wherein the capture probes have at least part of their sequence complementary to the miRNA sequences presented in table 1 and/or 2 and/or 3.

43. A kit of claim 41, wherein the capture probes have at least part of their sequence identical to the miRNA sequences presented in table 1 and/or 2 and/or 3.

44. A kit of claim 41, wherein the capture probes have a spacer being preferably located at a distance of 6.8 nm from the support and even being preferably a sequence of nucleotides being at least 20 bases and preferably more than 90 bases.

45. A kit for the determination of miRNA mediated cellular transcriptional regulation in a sample comprising two arrays comprising at least 3 capture probes being arranged in specific locations and reflecting the genomic or transcriptional matter of a cell, wherein the first array is dedicated to the detection and of multiple miRNAs present and the second array is dedicated to the detection and quantification of the expression of the regulated genes in the same sample and optionally, buffers and labels.

46. A kit of claim 45, wherein the two arrays are present on the same support.

47. A kit of claim 45, wherein the two arrays are present on the different supports.

48. A kit of claim 41, wherein the capture probes of the array for the detection of the miRNAs are nucleotide sequences having part of their sequence at least 90% homologous to the mRNA.

49. A kit of claim 45, wherein the capture probes of the array for the detection of the miRNAs are nucleotide sequences having part of their sequence at least 90% homologous to the mRNA.


SEQUENCE LISTING (239 sequences)

SEQ ID NO: 1 - DNA, 22 bp, Homo sapiens
aactatacaacctactacctca

SEQ ID NO: 2 - DNA, 22 bp, Homo sapiens
aaccacacaacctactacctca

SEQ ID NO: 3 - DNA, 21 bp, Homo sapiens
actatacaacctcctacctca

SEQ ID NO: 4 - DNA, 22 bp, Homo sapiens
acaaattcggttctacagggta

SEQ ID NO: 5 - DNA, 22 bp, Homo sapiens
acaaagttctgtagtgcactga

SEQ ID NO: 6 - DNA, 22 bp, Homo sapiens
gcaaaaatgtgctagtgccaaa

SEQ ID NO: 7 - DNA, 23 bp, Homo sapiens
cagtgaattctaccagtgccata

SEQ ID NO: 8 - DNA, 21 bp, Homo sapiens
ggctgtcaattcataggtcag

SEQ ID NO: 9 - DNA, 21 bp, Homo sapiens
gtctgtcaattcataggtcat

SEQ ID NO: 10 - DNA, 22 bp, Homo sapiens
aggcataggatgacaaagggaa

SEQ ID NO: 11 - DNA, 23 bp, Homo sapiens
cacaggttaaagggtctcaggga

SEQ ID NO: 12 - DNA, 21 bp, Homo sapiens
tacatacttctttacattcca

SEQ ID NO: 13 - DNA, 22 bp, Homo sapiens
cgcaaggtcggttctacgggtg

SEQ ID NO: 14 - DNA, 21 bp, Homo sapiens
acaggattgagggggggccct

SEQ ID NO: 15 - DNA, 21 bp, Homo sapiens
actttcggttatctagcttta

SEQ ID NO: 16 - DNA, 21 bp, Homo sapiens
acctatcctgaattacttgaa

SEQ ID NO: 17 - RNA, 21 nt, Homo sapiens
uggaagacuagugauuuuguu

SEQ ID NO: 18 - RNA, 21 nt, Homo sapiens
uggaagacuagugauuuuguu

SEQ ID NO: 19 - RNA, 21 nt, Homo sapiens
uggaagacuagugauuuuguu

SEQ ID NO: 20 - RNA, 22 nt, Homo sapiens
ugagguaguagauuguauaguu

SEQ ID NO: 21 - RNA, 22 nt, Homo sapiens
ugagguaguagauuguauaguu

SEQ ID NO: 22 - RNA, 21 nt, Homo sapiens
ugagguaggagguuguauagu

SEQ ID NO: 23 - RNA, 22 nt, Homo sapiens
ugagguaguagguuguauaguu

SEQ ID NO: 24 - RNA, 22 nt, Homo sapiens
ugagguaguagguuguauaguu

SEQ ID NO: 25 - RNA, 22 nt, Homo sapiens
ugagguaguagguuguauaguu

SEQ ID NO: 26 - RNA, 22 nt, Homo sapiens
ugagguaguagguugugugguu

SEQ ID NO: 27 - RNA, 22 nt, Homo sapiens
ugagguaguagguuguaugguu

SEQ ID NO: 28 - RNA, 21 nt, Homo sapiens
agagguaguagguugcauagu

SEQ ID NO: 29 - RNA, 23 nt, Homo sapiens
uacccuguagauccgaauuugug

SEQ ID NO: 30 - RNA, 22 nt, Homo sapiens
uacccuguagaaccgaauuugu

SEQ ID NO: 31 - RNA, 22 nt, Homo sapiens
uagcagcacauaaugguuugug

SEQ ID NO: 32 - RNA, 22 nt, Homo sapiens
uagcagcacguaaauauuggcg

SEQ ID NO: 33 - RNA, 20 nt, Homo sapiens
acugcagugaaggcacuugu

SEQ ID NO: 34 - RNA, 22 nt, Homo sapiens
uaaggugcaucuagugcagaua

SEQ ID NO: 35 - RNA, 23 nt, Homo sapiens
ugugcaaaucuaugcaaaacuga

SEQ ID NO: 36 - RNA, 23 nt, Homo sapiens
ugugcaaauccaugcaaaacuga

SEQ ID NO: 37 - RNA, 23 nt, Homo sapiens
ugugcaaauccaugcaaaacuga

SEQ ID NO: 38 - RNA, 22 nt, Homo sapiens
uaaagugcuuauagugcaggua

SEQ ID NO: 39 - RNA, 22 nt, Homo sapiens
uagcuuaucagacugauguuga

SEQ ID NO: 40 - RNA, 22 nt, Homo sapiens
aagcugccaguugaagaacugu

SEQ ID NO: 41 - RNA, 21 nt, Homo sapiens
aucacauugccagggauuucc

SEQ ID NO: 42 - RNA, 22 nt, Homo sapiens
uggcucaguucagcaggaacag

SEQ ID NO: 43 - RNA, 22 nt, Homo sapiens
uggcucaguucagcaggaacag

SEQ ID NO: 44 - RNA, 22 nt, Homo sapiens
cauugcacuugucucggucuga

SEQ ID NO: 45 - RNA, 22 nt, Homo sapiens
uucaaguaauccaggauaggcu

SEQ ID NO: 46 - RNA, 21 nt, Homo sapiens
uucaaguaauucaggauaggu

SEQ ID NO: 47 - RNA, 22 nt, Homo sapiens
uucacaguggcuaaguuccgcc

SEQ ID NO: 48 - RNA, 22 nt, Homo sapiens
aaggagcucacagucuauugag

SEQ ID NO: 49 - RNA, 22 nt, Homo sapiens
cuagcaccaucugaaaucgguu

SEQ ID NO: 50 - RNA, 23 nt, Homo sapiens
uguaaacauccuacacucucagc

SEQ ID NO: 51 - RNA, 22 nt, Homo sapiens
uguaaacauccccgacuggaag

SEQ ID NO: 52 - RNA, 23 nt, Homo sapiens
uguaaacauccucgacuggaagc

SEQ ID NO: 53 - RNA, 22 nt, Homo sapiens
cuuucagucggauguuugcagc

SEQ ID NO: 54 - RNA, 21 nt, Homo sapiens
ggcaagaugcuggcauagcug

SEQ ID NO: 55 - RNA, 21 nt, Homo sapiens
uauugcacauuacuaaguugc

SEQ ID NO: 56 - RNA, 19 nt, Homo sapiens
gugcauuguaguugcauug

SEQ ID NO: 57 - RNA, 22 nt, Homo sapiens
uggcagugucuuagcugguugu

SEQ ID NO: 58 - RNA, 24 nt, Homo sapiens
caaagugcuuacagugcagguagu

SEQ ID NO: 59 - RNA, 20 nt, Homo sapiens
acugcagugaaggcacuugu

SEQ ID NO: 60 - RNA, 22 nt, Homo sapiens
uauugcacuugucccggccugu

SEQ ID NO: 61 - RNA, 22 nt, Homo sapiens
uauugcacuugucccggccugu

SEQ ID NO: 62 - RNA, 22 nt, Homo sapiens
aaagugcuguucgugcagguag

SEQ ID NO: 63 - RNA, 22 nt, Homo sapiens
aaagugcuguucgugcagguag

SEQ ID NO: 64 - RNA, 22 nt, Homo sapiens
uucaacggguauuuauugagca

SEQ ID NO: 65 - RNA, 22 nt, Homo sapiens
uuuggcacuagcacauuuuugc

SEQ ID NO: 66 - RNA, 22 nt, Homo sapiens
ugagguaguaaguuguauuguu

SEQ ID NO: 67 - RNA, 22 nt, Homo sapiens
aacccguagauccgaucuugug

SEQ ID NO: 68 - RNA, 22 nt, Homo sapiens
aacccguagauccgaacuugug

SEQ ID NO: 69 - RNA, 22 nt, Homo sapiens
uacaguacugugauaacugaag

SEQ ID NO: 70 - RNA, 20 nt, Homo sapiens
uagcaccauuugaaaucagu

SEQ ID NO: 71 - RNA, 20 nt, Homo sapiens
uagcaccauuugaaaucagu

SEQ ID NO: 72 - RNA, 20 nt, Homo sapiens
uagcaccauuugaaaucagu

SEQ ID NO: 73 - RNA, 23 nt, Homo sapiens
agcaacauuguacagggcuauga

SEQ ID NO: 74 - RNA, 23 nt, Homo sapiens
agcagcauuguacagggcuauga

SEQ ID NO: 75 - RNA, 22 nt, Homo sapiens
ucaacaucagucugauaagcua

SEQ ID NO: 76 - RNA, 20 nt, Homo sapiens
ucaaaugcucagacuccugu

SEQ ID NO: 77 - RNA, 20 nt, Homo sapiens
ucaaaugcucagacuccugu

SEQ ID NO: 78 - RNA, 24 nt, Homo sapiens
aaaagugcuuacagugcagguagc

SEQ ID NO: 79 - RNA, 23 nt, Homo sapiens
agcagcauuguacagggcuauca

SEQ ID NO: 80 - RNA, 20 nt, Homo sapiens
uuaaggcacgcggugaaugc

SEQ ID NO: 81 - RNA, 18 nt, Homo sapiens
ucuacagugcacgugucu

SEQ ID NO: 82 - RNA, 20 nt, Homo sapiens
guguguggaaaugcuucugc

SEQ ID NO: 83 - RNA, 22 nt, Homo sapiens
ucagugcacuacagaacuuugu

SEQ ID NO: 84 - RNA, 22 nt, Homo sapiens
aacauucaaccugucggugagu

SEQ ID NO: 85 - RNA, 22 nt, Homo sapiens
accaucgaccguugauuguacc

SEQ ID NO: 86 - RNA, 23 nt, Homo sapiens
aacauucaacgcugucggugagu

SEQ ID NO: 87 - RNA, 21 nt, Homo sapiens
ugguucuagacuugccaacua

SEQ ID NO: 88 - RNA, 23 nt, Homo sapiens
uauggcacugguagaauucacug

SEQ ID NO: 89 - RNA, 21 nt, Homo sapiens
ucgugucuuguguugcagccg

SEQ ID NO: 90 - RNA, 21 nt, Homo sapiens
cugaccuaugaauugacagcc

SEQ ID NO: 91 - RNA, 22 nt, Homo sapiens
uagguaguuucauguuguuggg

SEQ ID NO: 92 - RNA, 22 nt, Homo sapiens
uagguaguuucauguuguuggg

SEQ ID NO: 93 - RNA, 21 nt, Homo sapiens
uagguaguuucauguuguugg

SEQ ID NO: 94 - RNA, 22 nt, Homo sapiens
uucaccaccuucuccacccagc

SEQ ID NO: 95 - RNA, 19 nt, Homo sapiens
gguccagaggggagauagg

SEQ ID NO: 96 - RNA, 23 nt, Homo sapiens
cccaguguucagacuaccuguuc

SEQ ID NO: 97 - RNA, 23 nt, Homo sapiens
cccaguguuuagacuaucuguuc

SEQ ID NO: 98 - RNA, 23 nt, Homo sapiens
cccaguguucagacuaccuguuc

SEQ ID NO: 99 - RNA, 22 nt, Homo sapiens
cccaguguucagacuaccuguu

SEQ ID NO: 100 - RNA, 24 nt, Homo sapiens
cucuaauacugccugguaaugaug

SEQ ID NO: 101 - RNA, 22 nt, Homo sapiens
gugaaauguuuaggaccacuag

SEQ ID NO: 102 - RNA, 22 nt, Homo sapiens
uucccuuugucauccuaugccu

SEQ ID NO: 103 - RNA, 22 nt, Homo sapiens
uccuucauuccaccggagucug

SEQ ID NO: 104 - RNA, 22 nt, Homo sapiens
auaagacgagcaaaaagcuugu

SEQ ID NO: 105 - RNA, 21 nt, Homo sapiens
cugugcgugugacagcggcug

SEQ ID NO: 106 - RNA, 22 nt, Homo sapiens
uucccuuugucauccuucgccu

SEQ ID NO: 107 - RNA, 21 nt, Homo sapiens
uaacagucuccagucacggcc

SEQ ID NO: 108 - RNA, 24 nt, Homo sapiens
aacauucauugcugucgguggguu

SEQ ID NO: 109 - RNA, 21 nt, Homo sapiens
acagcaggcacagacaggcag

SEQ ID NO: 110 - RNA, 21 nt, Homo sapiens
augaccuaugaauugacagac

SEQ ID NO: 111 - RNA, 21 nt, Homo sapiens
uaaucucagcuggcaacugug

SEQ ID NO: 112 - RNA, 24 nt, Homo sapiens
uacugcaucaggaacugauuggau

SEQ ID NO: 113 - RNA, 21 nt, Homo sapiens
uugugcuugaucuaaccaugu

SEQ ID NO: 114 - RNA, 21 nt, Homo sapiens
uugugcuugaucuaaccaugu

SEQ ID NO: 115 - RNA, 21 nt, Homo sapiens
ugauuguccaaacgcaauucu

SEQ ID NO: 116 - RNA, 21 nt, Homo sapiens
ccacaccguaucugacacuuu

SEQ ID NO: 117 - RNA, 23 nt, Homo sapiens
agcuacauugucugcuggguuuc

SEQ ID NO: 118 - RNA, 24 nt, Homo sapiens
agcuacaucuggcuacugggucuc

SEQ ID NO: 119 - RNA, 21 nt, Homo sapiens
ugucaguuugucaaauacccc

SEQ ID NO: 120 - RNA, 23 nt, Homo sapiens
caagucacuagugguuccguuua

SEQ ID NO: 121 - RNA, 22 nt, Mus musculus
uggaauguaaagaaguauguaa

SEQ ID NO: 122 - RNA, 22 nt, Mus musculus
uggaauguaaagaaguauguac

SEQ ID NO: 123 - RNA, 23 nt, Mus musculus
uggaauguaaagaaguauguauu

SEQ ID NO: 124 - RNA, 23 nt, Mus musculus
ucuuugguuaucuagcuguauga

SEQ ID NO: 125 - RNA, 21 nt, Mus musculus
uaaagcuagauaaccgaaagu

SEQ ID NO: 126 - RNA, 22 nt, Mus musculus
cccuguagaaccgaauuugugu

SEQ ID NO: 127 - RNA, 22 nt, Mus musculus
uagcagcacauaaugguuugug

SEQ ID NO: 128 - RNA, 22 nt, Mus musculus
uagcagcacaucaugguuuaca

SEQ ID NO: 129 - RNA, 22 nt, Mus musculus
uagcagcacguaaauauuggcg

SEQ ID NO: 130 - RNA, 22 nt, Mus musculus
uaaggugcaucuagugcagaua

SEQ ID NO: 131 - RNA, 23 nt, Mus musculus
ugugcaaauccaugcaaaacuga

SEQ ID NO: 132 - RNA, 23 nt, Mus musculus
uaaagugcuuauagugcagguag

SEQ ID NO: 133 - RNA, 22 nt, Mus musculus
uagcuuaucagacugauguuga

SEQ ID NO: 134 - RNA, 22 nt, Mus musculus
aagcugccaguugaagaacugu

SEQ ID NO: 135 - RNA, 21 nt, Mus musculus
aucacauugccagggauuucc

SEQ ID NO: 136 - RNA, 23 nt, Mus musculus
aucacauugccagggauuaccac

SEQ ID NO: 137 - RNA, 22 nt, Mus musculus
uggcucaguucagcaggaacag

SEQ ID NO: 138 - RNA, 22 nt, Mus musculus
uucaaguaauccaggauaggcu

SEQ ID NO: 139 - RNA, 22 nt, Mus musculus
uucaaguaauucaggauagguu

SEQ ID NO: 140 - RNA, 22 nt, Mus musculus
uucacaguggcuaaguuccgcu

SEQ ID NO: 141 - RNA, 20 nt, Mus musculus
uucacaguggcuaaguucug

SEQ ID NO: 142 - RNA, 22 nt, Mus musculus
cuagcaccaucugaaaucgguu

SEQ ID NO: 143 - RNA, 23 nt, Mus musculus
uagcaccauuugaaaucaguguu

SEQ ID NO: 144 - RNA, 22 nt, Mus musculus
uagcaccauuugaaaucgguua

SEQ ID NO: 145 - RNA, 23 nt, Mus musculus
uguaaacauccucgacuggaagc

SEQ ID NO: 146 - RNA, 22 nt, Mus musculus
cuuucagucggauguuugcagc

SEQ ID NO: 147 - RNA, 21 nt, Mus musculus
uguaaacauccuacacucagc

SEQ ID NO: 148 - RNA, 23 nt, Mus musculus
uguaaacauccuacacucucagc

SEQ ID NO: 149 - RNA, 22 nt, Mus musculus
uguaaacauccccgacuggaag

SEQ ID NO: 150 - RNA, 20 nt, Mus musculus
acccguagauccgaucuugu

SEQ ID NO: 151 - RNA, 22 nt, Mus musculus
cacccguagaaccgaccuugcg

SEQ ID NO: 152 - RNA, 20 nt, Mus musculus
uacaguacugugauaacuga

SEQ ID NO: 153 - RNA, 23 nt, Mus musculus
uggagugugacaaugguguuugu

SEQ ID NO: 154 - RNA, 23 nt, Mus musculus
uggagugugacaaugguguuuga

SEQ ID NO: 155 - RNA, 22 nt, Mus musculus
uuaaggcacgcggugaaugcca

SEQ ID NO: 156 - RNA, 21 nt, Mus musculus
uuaaggcacgcgggugaaugc

SEQ ID NO: 157 - RNA, 23 nt, Mus musculus
ucccugagacccuuuaaccugug

SEQ ID NO: 158 - RNA, 22 nt, Mus musculus
ucccugagacccuaacuuguga

SEQ ID NO: 159 - RNA, 21 nt, Mus musculus
ucguaccgugaguaauaaugc

SEQ ID NO: 160 - RNA, 21 nt, Mus musculus
cauuauuacuuuugguacgcg

SEQ ID NO: 161 - RNA, 22 nt, Mus musculus
ucggauccgucugagcuuggcu

SEQ ID NO: 162 - RNA, 22 nt, Mus musculus
ucacagugaaccggucucuuuu

SEQ ID NO: 163 - RNA, 21 nt, Mus musculus
cuuuuuucggucugggcuugc

SEQ ID NO: 164 - RNA, 22 nt, Mus musculus
cuuuuugcggucugggcuugcu

SEQ ID NO: 165 - RNA, 20 nt, Mus musculus
cagugcaauguuaaaagggc

SEQ ID NO: 166 - RNA, 23 nt, Mus musculus
uaacagucuacagccauggucgu

SEQ ID NO: 167 - RNA, 22 nt, Mus musculus
uugguccccuucaaccagcugu

SEQ ID NO: 168 - RNA, 22 nt, Mus musculus
ugugacugguugaccagaggga

SEQ ID NO: 169 - RNA, 24 nt, Mus musculus
uauggcuuuuuauuccuaugugaa

SEQ ID NO: 170 - RNA, 23 nt, Mus musculus
acuccauuuguuuugaugaugga

SEQ ID NO: 171 - RNA, 22 nt, Mus musculus
uauugcuuaagaauacgcguag

SEQ ID NO: 172 - RNA, 17 nt, Mus musculus
agcugguguugugaauc

SEQ ID NO: 173 - RNA, 18 nt, Mus musculus
ucuacagugcacgugucu

SEQ ID NO: 174 - RNA, 21 nt, Mus musculus
agugguuuuacccuaugguag

SEQ ID NO: 175 - RNA, 21 nt, Mus musculus
aacacugucugguaaagaugg

SEQ ID NO: 176 - RNA, 20 nt, Mus musculus
cauaaaguagaaagcacuac

SEQ ID NO: 177 - RNA, 22 nt, Mus musculus
uguaguguuuccuacuuuaugg

SEQ ID NO: 178 - RNA, 22 nt, Mus musculus
ugagaugaagcacuguagcuca

SEQ ID NO: 179 - RNA, 22 nt, Mus musculus
uacaguauagaugauguacuag

SEQ ID NO: 180 - RNA, 24 nt, Mus musculus
guccaguuuucccaggaaucccuu

SEQ ID NO: 181 - RNA, 23 nt, Mus musculus
ugagaacugaauuccauggguuu

SEQ ID NO: 182 - RNA, 21 nt, Mus musculus
guguguggaaaugcuucugcc

SEQ ID NO: 183 - RNA, 22 nt, Mus musculus
ucagugcacuacagaacuuugu

SEQ ID NO: 184 - RNA, 22 nt, Mus musculus
ucuggcuccgugucuucacucc

SEQ ID NO: 185 - RNA, 23 nt, Mus musculus
ucucccaacccuuguaccagugu

SEQ ID NO: 186 - RNA, 22 nt, Mus musculus
cuagacugaggcuccuugaggu

SEQ ID NO: 187 - RNA, 21 nt, Mus musculus
ucagugcaugacagaacuugg

SEQ ID NO: 188 - RNA, 20 nt, Mus musculus
uugcauagucacaaaaguga

SEQ ID NO: 189 - RNA, 22 nt, Mus musculus
uagguuauccguguugccuucg

SEQ ID NO: 190 - RNA, 22 nt, Mus musculus
uuaaugcuaauugugauagggg

SEQ ID NO: 191 - RNA, 23 nt, Mus musculus
aacauucaacgcugucggugagu

SEQ ID NO: 192 - RNA, 22 nt, Mus musculus
uuuggcaaugguagaacucaca

SEQ ID NO: 193 - RNA, 23 nt, Mus musculus
uauggcacugguagaauucacug

SEQ ID NO: 194 - RNA, 22 nt, Mus musculus
uggacggagaacugauaagggu

SEQ ID NO: 195 - RNA, 18 nt, Mus musculus
uggagagaaaggcaguuc

SEQ ID NO: 196 - RNA, 23 nt, Mus musculus
caaagaauucuccuuuugggcuu

SEQ ID NO: 197 - RNA, 22 nt, Mus musculus
ucgugucuuguguugcagccgg

SEQ ID NO: 198 - RNA, 22 nt, Mus musculus
caucccuugcaugguggagggu

SEQ ID NO: 199 - RNA, 23 nt, Mus musculus
gugccuacugagcugacaucagu

SEQ ID NO: 200 - RNA, 22 nt, Mus musculus
ugauauguuugauauauuaggu

SEQ ID NO: 201 - RNA, 22 nt, Mus musculus
caacggaaucccaaaagcagcu

SEQ ID NO: 202 - RNA, 18 nt, Mus musculus
cugaccuaugaauugaca

SEQ ID NO: 203 - RNA, 21 nt, Mus musculus
aacuggccuacaaagucccag

SEQ ID NO: 204 - RNA, 22 nt, Mus musculus
uguaacagcaacuccaugugga

SEQ ID NO: 205 - RNA, 21 nt, Mus musculus
uagcagcacagaaauauuggc

SEQ ID NO: 206 - RNA, 21 nt, Mus musculus
uagguaguuucauguuguugg

SEQ ID NO: 207 - RNA, 22 nt, Mus musculus
cccaguguucagacuaccuguu

SEQ ID NO: 208 - RNA, 22 nt, Mus musculus
uacaguagucugcacauugguu

SEQ ID NO: 209 - RNA, 22 nt, Mus musculus
uaacacugucugguaacgaugu

SEQ ID NO: 210 - RNA, 23 nt, Mus musculus
uaauacugccugguaaugaugac

SEQ ID NO: 211 - RNA, 21 nt, Mus musculus
uacucaguaaggcauuguucu

SEQ ID NO: 212 - RNA, 22 nt, Mus musculus
agagguauagcgcaugggaaga

SEQ ID NO: 213 - RNA, 23 nt, Mus musculus
gugaaauguuuaggaccacuaga

SEQ ID NO: 214 - RNA, 23 nt, Mus musculus
uucccuuugucauccuaugccug

SEQ ID NO: 215 - RNA, 22 nt, Mus musculus
uccuucauuccaccggagucug

SEQ ID NO: 216 - RNA, 22 nt, Mus musculus
uggaauguaaggaagugugugg

SEQ ID NO: 217 - RNA, 23 nt, Mus musculus
gcuucuccuggcucuccucccuc

SEQ ID NO: 218 - RNA, 22 nt, Mus musculus
auaagacgagcaaaaagcuugu

SEQ ID NO: 219 - RNA, 22 nt, Mus musculus
ugagguaguagguugugugguu

SEQ ID NO: 220 - RNA, 22 nt, Mus musculus
ugagguaguagguuguauaguu

SEQ ID NO: 221 - RNA, 22 nt, Mus musculus
ugagguaguagguuguaugguu

SEQ ID NO: 222 - RNA, 21 nt, Mus musculus
agagguaguagguugcauagu

SEQ ID NO: 223 - RNA, 21 nt, Mus musculus
ugagguaggagguuguauagu

SEQ ID NO: 224 - RNA, 22 nt, Mus musculus
ugagguaguagauuguauaguu

SEQ ID NO: 225 - RNA, 22 nt, Mus musculus
ugagguaguagauuguauaguu

SEQ ID NO: 226 - RNA, 22 nt, Mus musculus
ugagguaguaguuuguacagua

SEQ ID NO: 227 - RNA, 22 nt, Mus musculus
ugagguaguaguguguacaguu

SEQ ID NO: 228 - RNA, 19 nt, Mus musculus
ugagguaguaguuugugcu

SEQ ID NO: 229 - RNA, 22 nt, Homo sapiens
ugagguaguagguuguauaguu

SEQ ID NO: 230 - RNA, 22 nt, Homo sapiens
ugagguaguagguugugugguu

SEQ ID NO: 231 - RNA, 21 nt, Homo sapiens
ugagguaggagguuguauagu

SEQ ID NO: 232 - RNA, 22 nt, Homo sapiens
uacccuguagaaccgaauuugu

SEQ ID NO: 233 - RNA, 22 nt, Homo sapiens
uuuggcacuagcacauuuuugc

SEQ ID NO: 234 - RNA, 22 nt, Homo sapiens
ucagugcacuacagaacuuugu

SEQ ID NO: 235 - RNA, 23 nt, Homo sapiens
uauggcacugguagaauucacug

SEQ ID NO: 236 - RNA, 21 nt, Homo sapiens
cugaccuaugaauugacagcc

SEQ ID NO: 237 - RNA, 22 nt, Homo sapiens
uucccuuugucauccuaugccu

SEQ ID NO: 238 - RNA, 21 nt, Homo sapiens
augaccuaugaauugacagac

SEQ ID NO: 239 - RNA, 15 nt, Artificial Sequence
  Description of Artificial Sequence Synthetic poly A sequence
aaaaaaaaaaaaaaa